SEQUENCE LISTING (8 sequences)

SEQ ID NO: 1 - protein, 6 aa, synthetic construct
EGGGVR

SEQ ID NO: 2 - protein, 6 aa, synthetic construct
ADDDDK

SEQ ID NO: 3 - protein, 625 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 4 - DNA, 30 bp, synthetic construct
  source 1..30: mol_type = other DNA
cattgctcagcggtgctgtccatctgcacg

SEQ ID NO: 5 - DNA, 25 bp, synthetic construct
  source 1..25: mol_type = other DNA
ttccggtaccccagtctccagtgcc

SEQ ID NO: 6 - DNA, 26 bp, synthetic construct
  source 1..26: mol_type = other DNA
agtagttaacaggccataggtgaatt

SEQ ID NO: 7 - DNA, 26 bp, synthetic construct
  source 1..26: mol_type = other DNA
aagccagcagtactggctcctaatgt

SEQ ID NO: 8 - protein, 644 aa, synthetic construct
MFSMRIVCLVLSVVGTAWTADSGEGDFLAADDDDKGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

(19) United States
(12) Patent Application Publication
(10) Pub. No.: US 2025/0205313 A1
(43) Pub. Date: Jun. 26, 2025

(54) NONPOLYMERIZABLE FIBRINOGEN AS AN ANTITHROMBOTIC AGENT

(71) Applicant: The University of North Carolina at Chapel Hill, Chapel Hill, NC (US)

(72) Inventors: Matthew James Flick, Chapel Hill, NC (US); Alisa Sue Wolberg, Chapel Hill, NC (US); Wolfgang Bergmeier, Chapel Hill, NC (US); Nigel Mackman, Chapel Hill, NC (US); Woosuk Steve Hur, Carrboro, NC (US)

(21) Appl. No.: 18/989,581

(22) Filed: Dec. 20, 2024

Related U.S. Application Data
(60) Provisional application No. 63/612,524, filed on Dec. 20, 2023.

(51) Int. Cl. A61K 38/36; A61K 45/06; A61P 7/02; C07K 14/75

(52) U.S. Cl. CPC A61K 38/363; A61K 45/06; A61P 7/02; C07K 14/75

(57) ABSTRACT

This invention relates to methods for inhibiting or reducing the risk of thrombosis or reducing the size and number of thrombi without compromising hemostasis using a nonpolymerizable fibrinogen that is insensitive to thrombin cleavage. Methods and compositions for reducing a required dose or complementing the effect of an antithrombotic agent in the treatment of thrombosis are also provided.

### CROSS-REFERENCE TO RELATED APPLICATION

This application claims benefit from U.S. Provisional Patent Application Ser. No. 63/612,524, filed Dec. 20, 2023, the contents of which are incorporated herein by reference in their entireties.

### STATEMENT OF GOVERNMENT SUPPORT

This invention was made with government support under Grant Nos. DK112778 and HL168009 awarded by the National Institutes of Health. The government has certain rights in the invention.

### FIELD OF THE INVENTION

This invention relates to methods for inhibiting or reducing the risk of thrombosis or reducing the size and number of thrombi without compromising hemostasis using a nonpolymerizable fibrinogen that is insensitive to thrombin cleavage. Methods and compositions for reducing a required dose or complementing the effect of an antithrombotic agent in the treatment of thrombosis are also provided.

### STATEMENT REGARDING ELECTRONIC FILING OF A SEQUENCE LISTING

A Sequence Listing in XML format, entitled 5470-967_ST26.xml, 9,205 bytes in size, generated on Dec. 3, 2024, is filed herewith. This Sequence Listing is hereby incorporated herein by reference into the specification for its disclosures.

### BACKGROUND OF THE INVENTION

Thrombotic diseases are the leading cause of death in the United States (Wendelboe & Raskob (2016) Circ. Res. 118(9):1340-1347). Cardiovascular disease and arterial thrombosis (AT) accounted for approximately 20% of deaths in 2020 (Ahmad & Anderson (2021) JAMA 325(18):1829-1830), and are the leading cause of quality-of-life reduction (Halimeh et al. (2022) Hamostaseologie 42(2):123-130; Shaydakov et al. (2022) J. Vasc. Surg. Venous Lymphat. Disord. 10(1):241-247; Amirtabar et al. (2023) J. Thromb. Thrombolysis 55(1):185-188). Venous thromboembolism (VTE) affects approximately 900,000 people annually in the US, resulting in up to 100,000 deaths (Heit (2015) Nat. Rev. Cardiol. 12(8):464-474). Different mechanisms drive arterial and venous thrombosis. In AT, atherosclerotic plaque rupture and subsequent sub-endothelium exposure induce platelet aggregation and activation, leading to the formation of a platelet-rich thrombus (Chernysh et al. (2020) Sci. Rep. 10(1):5112). In VTE, stasis of blood flow activates endothelial cells, triggers inflammation and initiates the coagulation cascade, leading to a fibrin- and red blood cell (RBC)-rich thrombus (Chernysh et al. (2020) Sci. Rep. 10(1):5112). Despite unique modes of pathogenesis, quantitative and qualitative differences in fibrin(ogen) are major determinants of both arterial and venous thrombosis.

Activation of coagulation results in thrombin-mediated cleavage of monomeric fibrinogen to fibrin that self-polymerizes to form a fibrin matrix. In healthy individuals, fibrinogen circulates in plasma at a concentration of 2-4 mg/mL. Elevated circulating fibrinogen levels positively correlate with thrombosis risk in humans and mice (Machlus et al. (2011) Blood 117(18):4953-4963). Hyperfibrinogenemia promotes increased coagulability and the formation of a dense and fibrinolysis-resistant fibrin matrix (Machlus et al. (2011) Blood 117(18):4953-4963; Wolberg (2007) Blood Rev. 21(3):131-142; Collet et al. (2000) Arterioscler. Thromb. Vasc. Biol. 20(5):1354-1361; Kim et al. (2007) J. Thromb. Haemost. 5(6):1250-1256). Dense fibrin matrices with highly branched fibrils support robust RBC retention and suppress platelet/fibrin-mediated clot contraction, resulting in delayed clot clearance (Aleman et al. (2014) J. Clin. Invest. 124(8):3590-3600; Byrnes et al. (2015) Blood 126(16):1940-1948). Conversely, genetically-imposed hypofibrinogenemia or pharmacological reduction of circulating fibrinogen significantly reduces experimental venous thrombus formation in mice (Hur et al. (2022) Blood 139(9):1374-1388).

The present invention addresses an unmet need by providing an improved therapy for inhibiting thrombosis without compromising hemostasis.

### SUMMARY OF THE INVENTION

This invention is based on the finding that nonpolymerizable fibrinogen differentially suppresses arterial and venous thrombosis while markedly preserving hemostatic potential. Results presented herein indicate that the presence of a nonpolymerizable fibrinogen in the milieu with normal fibrinogen suppressed fibrin matrix formation leading to a significant reduction in thrombosis incidence and thrombus size. Selective prevention of fibrin matrix formation using nonpolymerizable fibrinogen provides a therapeutic strategy against thrombosis without a significant increase in bleeding risk. Such a strategy is of use in pathological conditions where venous thrombosis is a significant co-morbidity (e.g., cancer, obesity, inflammatory diseases, and metabolic syndrome). Furthermore, a nonpolymerizable fibrinogen provides combined beneficial effects with antiplatelet therapies for conditions in which arterial thrombosis is a significant risk factor (e.g., obesity and metabolic syndrome).

Thus, one aspect of the invention relates to a method for inhibiting or reducing the risk of venous or arterial thrombosis without compromising hemostasis, comprising administering to a subject in need thereof an effective amount of nonpolymerizable fibrinogen thereby inhibiting or reducing the risk of venous or arterial thrombosis without compromising hemostasis.

Another aspect of the invention relates to a method of reducing the number and/or size of thrombi formed in a subject without compromising hemostasis, comprising administering to a subject in need thereof an effective amount of nonpolymerizable fibrinogen thereby reducing the number and/or size of thrombi formed in the subject without compromising hemostasis.

A further aspect of the invention relates to a method of reducing a required dose or complementing the effect of an antithrombotic agent in the treatment of thrombosis in a subject in need thereof, comprising administering to the subject an effective amount of a nonpolymerizable fibrinogen in combination with the antithrombotic agent.

Another aspect of the invention relates to a pharmaceutical composition comprising an effective amount of a nonpolymerizable fibrinogen and an antithrombotic agent in admixture with a pharmaceutically acceptable carrier.

These and other aspects of the invention are set forth in more detail in the description of the invention below.

### BRIEF DESCRIPTION OF THE DRAWINGS

FIG. 1 shows that fibrinogen^{LOCK} limits thrombus formation. Fibrinogen^{LOCK} terminates fibrin fibril elongation (top) in a manner analogous to dideoxynucleotide termination of DNA polymerization (bottom).

FIGS. 2A-2J show that Fga^{WT/EK} and Fga^{EK/EK} mice are protected against FeCl₃-induced model of arterial thrombosis depending on injury severity. (FIG. 2A) Representative traces of blood flow, (FIG. 2B) vessel occlusion times and (FIG. 2C) embolic incidence per mouse following 3-minute exposure of 4% FeCl₃ in Fga^{WT/WT}, Fga^{WT/EK}, Fga^{EK/EK} and Fga^{+/-} mice. (FIG. 2D) Representative traces of blood flow, (FIG. 2E) vessel occlusion times and (FIG. 2F) embolic incidence per mouse following 3-minute exposure of 5% FeCl₃ in Fga^{WT/WT} Fga^{WT/EK}, Fga^{EK/EK} and Fga^{+/-} mice. (FIG. 2G) Representative traces of blood flow, (FIG. 2H) vessel occlusion times and (FIG. 2I) embolic incidence per mouse following 3-minute exposure of 10% FeCl₃ in Fga^{WT/WT}, Fga^{WT/EK}, Fga^{EK/EK}, Fga^{+/-} and Fga^{-/-} mice. Open arrows indicate embolic events. Horizontal bars indicate the median. Data were analyzed using Kruskal-Wallis test with ns: not significant, *p<0.05, and ***p<0.001. (FIG. 2J) Representative images of platelet accumulation in the carotid arteries following injury with 8% FeCl₃ for 1 minute.

FIGS. 3A-3H show that platelet aggregation and intraplatelet levels of fibrinogen and fibronectin are unaltered in Fga^{EK} mice. Aggregation traces of platelet-rich plasma collected from Fga^{WT/WT}, Fga^{WT/EK}, Fga^{EK/EK} and Fga^{-/-} mice following stimulation with (FIG. 3A) 5 μM ADP (n=4/genotype) or (FIG. 3B) 250 μM protease-activated receptor 4 activating peptide (Par4p, n=4/genotype). (FIG. 3C) Western blot analyses for fibrinogen, fibronectin, and actin of platelet lysates harvested from Fga^{WT/WT}, Fga^{WT/EK} and Fga^{EK/EK} mice. Quantification of platelet (FIG. 3D) fibrinogen and (FIG. 3E) fibronectin from the western blot analyses. (FIG. 3F) Western blot analyses for fibrinogen, fibronectin, and albumin of plasma harvested from Fga^{WT/WT}, Fga^{WT/EK} and Fga^{EK/EK} mice (n=3/genotype). Quantification of plasma (FIG. 3G) fibrinogen and (FIG. 3H) fibronectin from the western blot analyses. Data are presented as the mean±SEM and analyzed using one-way ANOVA with ns: not significant.

FIGS. 4A-4E show that Fga^{EK/EK} and Fga^{WT/EK} mice are protected from venous thrombosis. (FIG. 4A) Incidence of thrombus formation and (FIG. 4B) thrombus weights from Fga^{WT/WT}, Fga^{WT/EK} and Fga^{EK/EK} mice 24 hr after inferior vena cava (IVC) ligation (stasis model). (FIG. 4C) Circulating fibrinogen levels of before and after IVC ligation measured by ELISA. (FIG. 4D) Spearman correlation analysis of circulating fibrinogen level post-ligation and thrombus mass. Data are presented as the mean±SEM and analyzed using Kruskal-Wallis test with ns: not significant, **p<0.01, and ***p<0.001. (FIG. 4E) Representative images of 5 μm-thick sections of thrombi stained against fibrin(ogen) (asterisk), CD41 (arrowhead) and DAPI (open arrow). Note that Fga^{EK/EK} thrombi contained minimal platelets and nucleated cells, as well as morphologically distinct aggregates of fibrinogen, compared to Fga^{WT/WT} and Fga^{WT/EK} thrombi that display web-like fibrin matrices.

FIGS. 5A-5D show that Fga^{WT/EK} and Fga^{EK/EK} whole blood have reduced or eliminated clot formation in vitro, respectively. (FIG. 5A) Mean TEG curve tracings of whole blood activated with tissue factor and calcium. (FIG. 5B) Maximum amplitude (MA), (FIG. 5C) a angle and (FIG. 5D) clot initiation time (R) of whole blood isolated from Fga^{WT/WT}, Fga^{WT/EK}, Fga^{EK/EK}, Fga^{+/-} and Fga^{-/-} mice.

FIGS. 6A-6H show that Fga^{WT/EK} clots have comparable mechanical strength and are resistant to lysis that is not accounted for by differences in clot contraction or FXIII activation. (FIG. 6A) Mean TEG curve tracings of whole blood activated with tissue factor and calcium in the presence of tPA. (FIG. 6B) Maximum amplitude (MA), (FIG. 6C) α angle, (FIG. 6D) R time and (FIG. 6E) lysis at 30 min (LY30) of whole blood isolated from Fga^{WT/WT}, Fga^{WT/EK} and Fga^{+/-} mice. (FIG. 6F) Clot mass following clot retraction. (FIG. 6G) Representative western blots and (FIG. 6H) quantification (n=3/genotype) of FXIIIa during time course of thrombin-initiated clotting in Fga^{WT/WT}, Fga^{WT/EK} and Fga^{EK/EK} plasma.

FIGS. 7A-7D show that nonpolymerizable fibrinogen functions as a chain terminating agent. (FIG. 7A) Representative SEM images of clots made with platelet-poor plasma of Fga^{WT/WT} and Fga^{WT/EK} mice. Arrows indicate fiber termini. (FIG. 7B) Diameters of fibrin fibers of Fga^{WT/WT} and Fga^{WT/EK} clots. Large dots indicate biological replicates and small dots indicate total fibers analyzed from all samples. (FIG. 7C) Quantification of the number of fiber terminal ends per fiber in clots made from Fga^{WT/WT} and Fga^{WT/EK} mice. (FIG. 7D) Fibrin turbidity, where Ca²⁺/Thrombin was added to mixtures of platelet-poor plasma collected from Fga^{WT/WT} and Fga^{EK/EK} at different ratios of fibrinogen^{WT} and fibrinogen^{LOCK}. These data show that fibrinogen^{LOCK} limits fibrin formation in vitro. Data are presented as the mean±SEM and analyzed using one-way ANOVA with ns: not significant, *p<0.05, **p<0.01, ***p<0.001.

FIGS. 8A-8H show that nonpolymerizable fibrinogen^{EK} limits fibrin formation and suppresses fibrinolysis in the small clots that do form. (FIG. 8A-8B) Turbidity analysis of Fga^{WT/WT}, Fga^{WT/EK}, Fga^{EK/EK}, Fga^{+/-} and Fga^{-/-} plasma (n=3) in the (FIG. 8A) absence and (FIG. 8B) presence of tPA. (FIG. 8C-8D) Turbidity analysis of plasma containing different ratios of Fga^{WT/WT} and Fga^{EK/EK} plasma (n=3/genotype) in the (FIG. 8C) absence and (FIG. 8D) presence of tPA. (FIG. 8E-8F) Turbidity analysis using increasing amount of purified Fib^{WT} reconstituted (0.5-1.0 mg/mL) in Fga^{EK/EK} plasma (n=2) in the (FIG. 8E) absence and (FIG. 8F) presence of tPA. (FIG. 8G-8H) Turbidity analysis using purified Fib^{WT} (0.5 mg/mL) and increasing amounts of purified Fib^{EK} (0-0.5 mg/mL) reconstituted in Fga^{-/-} plasma (n=2) in the (FIG. 8G) absence and (FIG. 8H) presence of tPA.

FIGS. 9A-9F show that Fga^{EK/EK} mice have preserved hemostatic potential. (FIG. 9A) Time to cessation of bleeding (sustained >30 seconds) of Fga^{WT/WT}, Fga^{WT/EK}, Fga^{EK/EK} Fga^{+/-} and Fga^{-/-} mice following 3 mm excision of the distal portion of the tail. Horizontal bar indicates the mean with data analyzed using the Kaplan-Meier log-rank test with ns: not significant, **p<0.01, and ***p<0.001. (FIG. 9B) Time to cessation of bleeding of Fga^{WT/WT}, Fga^{WT/EK}, Fga^{EK/EK} and Fga^{-/-} mice following laser-induced saphenous vein injury (n=18-23/genotype). Horizontal Bar indicates the mean with data analyzed using one-way ANOVA with ***p<0.001. (FIG. 9C) Representative images of platelet plug and fibrin(ogen) 5 minutes following laser-induced saphenous vein injury (FIG. 9D). Representative 3-dimensional reconstruction of injury sites depicting the side view following injury. Each grid box=50 μm □ 50 μm. Quantification of (FIG. 9E) fibrin(ogen) and (FIG. 9F) platelet accumulation at the site of injury. Data are expressed as the mean±SEM and analyzed using one-way ANOVA with *p<0.05.

FIGS. 10A-10C show that Fga^{WT/EK} and Fga^{EK/EK} mice have normal wound healing. (FIG. 10A) Representative images of wound fields and (FIG. 10B) plot of the percentage of mice healed following 1-cm surgical incision in Fga^{WT/WT} (n=6), Fga^{WT/EK} (n=8), Fga^{EK/EK} (n=6), and Fga^{-/-} (n=6) mice. Data were analyzed using Kaplan-Meier log-rank test with p<0.05 for Fga^{WT/WT} mice vs. Fga^{-/-} mice and for Fga^{WT/EK} mice vs. Fga^{-/-} mice. (FIG. 10C) Representative H&E stain of 5-μm thick sagittal sections of wound site of Fga^{WT/WT} Fga^{WT/EK}, Fga^{EK/EK} and Fga^{-/-} mice that healed 15 days after incision. Scale bar: 200 μm.

FIG. 11 shows LPS-induced inflammation provokes a significant increase in circulating fibrinogen in Fga^{WT/WT}, Fga^{WT/EK}, and Fga^{EK/EK} mice. Shown are the results of ELISA for fibrinogen on plasma isolated from mice 24 hours after induction of endotoxemia.

FIGS. 12A-12E show that Fga^{WT/WT}, Fga^{WT/EK} and Fga^{EK/EK} mice display the same reactive changes in blood cells after LPS-induced inflammation. Complete Blood Cell (CBC) counts, including white blood cells (WBC, FIG. 12A), neutrophils (Neu, FIG. 12B), lymphocytes (Lym, FIG. 12C), red blood cells (RBC, FIG. 12D), and platelets (PLT, FIG. 12E), were carried out on whole blood isolated from mice 24 hours after induction of endotoxemia.

FIGS. 13A-13C show that Fga^{WT/EK} and Fga^{EK/EK} mice are protected from LPS-driven thrombosis. Mice were subjected of inferior vena cave thrombosis induced by 10% FeCl₃ exposure 24 hours after LPS administration. Thrombosis weight (FIG. 13A), thrombosis incidence (FIG. 13B) and weight loss (FIG. 13C) were assessed.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.831-1.835 and established usage. See, e.g., WIPO Standard ST.26. WIPO Handbook on Intellectual Property Information and Documentation.

Except as otherwise indicated, standard methods known to those skilled in the art may be used for the construction of recombinant parvovirus and AAV (rAAV) constructs, packaging vectors expressing the parvovirus Rep and/or Cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, NY, 2012); AUSUBEL et al., CURRENT PROTOCOLS IN MOLECULAR BIOLOGY (Green Publishing Associates, Inc. and John Wiley & Sons, Inc., New York).

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, 5%, 1%, 0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Pharmaceutically acceptable carrier” (sometimes referred to as a “carrier”) refers to a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005).

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

“Inhibit” or “reduce” or grammatical variations thereof as used herein refers to a decrease or diminishment in the specified level or activity of at least about 1%, 5%, 10%, 15%, 25%, 35%, 40%, 50%, 60%, 75%, 80%, 90%, 95% or more. In particular embodiments, the inhibition or reduction results in little or essentially no detectible activity or amount (at most, an insignificant amount, e.g., less than about 10% or even 5%).

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

Grammatical variations of“administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Subjects” according to the present invention include mammals, avians, reptiles, amphibians, and fish. Mammalian subjects include but are not limited to humans, non-human mammals, non-human primates (e.g., monkeys, chimpanzees, baboons, etc.), dogs, cats, mice, hamsters, rats, horses, cows, pigs, rabbits, sheep and goats. In some embodiments, the subject is a laboratory animal. Human subjects include neonates, infants, juveniles, adults, and geriatric subjects. In some embodiments, the subject in need of treatment may include those at risk of thrombosis or developing thrombi. A subject having an increased risk of thrombosis may include a patient receiving transplanted cells, tissues or organs including hematopoietic transplants, bone marrow transplants, kidney, heart, liver, lung and the like as well as patients receiving certain therapies such as chemotherapy or radiation thereby, surgery or extracorporeal membrane oxygenation (ECMO) treatment.

“Thrombosis” refers to the formation of a thrombus, meaning a blood clot comprising platelets, fibrin, leukocytes, and red blood cells located within a vascular lumen (Rubin's Pathology, Raphael Rubin and David S. Strayer, ed., 5th Ed., Lippincott Williams & Wilkins: 2008, page 233). The term encompasses arterial and venous thrombosis, including deep vein thrombosis, portal vein thrombosis, jugular vein thrombosis, renal vein thrombosis, stroke, myocardial infarction, Budd-Chiari syndrome, Paget-Schroetter disease, and cerebral venous sinus thrombosis. A thrombus is distinct from a typical blood clot. While a blood clot results from activation of the coagulation cascade, a thrombus also involves adherence and aggregation of platelets, participation of cellular elements of the immune system, and active participation of endothelial cells of the blood vessel.

Before injury to a blood vessel, circulating platelets are in a nonadherent state. Injury activates platelet adhesiveness, after which platelets bind to one another to form an aggregate of activated platelets (platelet thrombus). These platelet aggregates occlude injured small vessels and prevent leakage of blood. Once platelets are stimulated to adhere to the vessel wall, their granular contents are released, in part by contraction of the platelet cytoskeleton. In turn, these granules promote aggregation of other platelets. Platelet adhesion is enhanced by release of subendothelial von Willebrand factor, which is adhesive for Gp1b platelet membrane protein and for fibrinogen. Activated platelets also release ADP and thromboxane A2, which recruit additional platelets to the process. The platelet membrane protein complex GpIIb-IIIc binds to fibrinogen, thereby forming fibrinogen bridges between platelets, enhancing aggregation, and stabilizing the nascent thrombus. Activated platelets in turn release factors that initiate coagulation, thus forming a complex thrombus on the vessel wall. Thrombin itself stimulates further release of platelet granules and subsequent recruitment of new platelets.

As used herein, “venous thrombosis” refers to a thrombus within a vein. The pathogenesis of venous thrombosis is inherently tied to fibrin matrix formation. Studies have suggested that the configuration of the fibrin matrix itself is a major determinant of clot formation, stability and ultimately venous thrombosis (Machlus et al. (2011) Blood 117(18):4953-4963; Aleman et al. (2014) J. Clin. Invest. 124(8):3590-3600; Byrnes et al. (2015) Blood 126(16):1940-1948; Aleman et al. (2013) Arterioscler. Thromb. Vasc. Biol. 33(8):1829-1836). High thrombin and fibrinogen concentrations result in the formation of thin fibrin fibers in a highly dense matrix that promotes RBC retention and suppresses platelet/fibrin-mediated clot contraction, leading to delayed clearance (Aleman et al. (2014) J. Clin. Invest. 124(8):3590-3600; Aleman et al. (2013) Arterioscler. Thromb. Vasc. Biol. 33(8):1829-1836; Wolberg et al. (2010) Thromb. Res. 125(Suppl 1):S35-37). FXIIIa crosslinks fibrin α-chains (Byrnes et al. (2015) Blood 126(16):1940-1948) to establish this dense matrix composed of more elastic fibrin fibers that promote RBC retention and subsequently larger thrombi.

Risk factors for venous thrombosis include, without limitation, surgery; trauma; immobilization; previous thrombosis; cancer; pregnancy; antiphospholipid antibodies syndrome; medical conditions such as atherosclerosis, heart failure, hypertension, dyslipidemia, chronic kidney disease, renal transplant, nephrotic syndrome, microalbuminuria, polycythemia vera, paroxysmal nocturnal hemoglobinuria, hyperhomocysteinemia, Behcet disease, rheumatoid arthritis, systemic lupus erythematosus, antineutrophil cytoplasmic antibodies-associated vasculitis, inflammatory bowel disease, sepsis, coronavirus disease 2019, tuberculosis, asthma, obstructive sleep apnea, polycystic ovary syndrome, diabetes mellitus; and an inherited hypercoagulable disorder (Factor V Leiden mutation, prothrombin gene mutation, protein C deficiency, protein S deficiency, antithrombin deficiency, dysfibrinogenemia, factor XII deficiency, or hyperhomocysteinemia).

As used herein, “arterial thrombosis” refers to a thrombus within an artery. Arterial thrombosis is mediated by platelets, which serve as the primary driver of thrombus growth (i.e., arterial thrombi are platelet-rich). The coronary, cerebral, mesenteric, and renal arteries, and arteries of the lower extremities, are the vessels most commonly involved in an arterial thrombosis due to atherosclerosis. Arterial thrombosis may also occur, however, as a result of other disorders, including inflammation of arteries (arteritis), trauma, and blood diseases. Thrombi are also common in aneurysms (localized-dilations of the lumen) of the aorta and its major branches, in which the distortion of blood flow, combined with intrinsic vascular disease, promotes thrombosis.

Risk factors for thrombosis in the arterial system include, without limitation, immobilization after surgery or leg casting, obesity, advanced age, previous thrombosis, and cancer. The three factors that are commonly associated with development of thrombosis are: (1) damage to the endothelium, usually by atherosclerosis, which disturbs the anticoagulant properties of the vessel wall and serves as a site of origin for platelet aggregation and fibrin formation; (2) alteration in blood flow, whether from turbulence at the site of an aneurysm, sites of arterial bifurcation, or slowing of blood flow in narrowed arteries; and (3) increased coagulability of the blood. Since most arterial thrombi occlude the vessel in which they occur, they often lead to ischemic necrosis of tissue supplied by that artery, i.e., an infarct. Infarction is the process by which coagulative necrosis develops in an area distal to the occlusion of an end-artery. Thrombosis of a coronary or cerebral artery results in myocardial infarct (heart attack) or cerebral infarct (stroke), respectively.

“Deep vein thrombosis” refers to a thrombus that develops in a deep vein, usually in the lower leg. Deep venous thrombosis often results from one or more of the same causative factors that favor arterial and cardiac thrombosis. Those factors are endothelial injury (e.g., trauma, surgery, childbirth), stasis (e.g., heart failure, chronic venous insufficiency, post-operative immobilization, prolonged bed rest) and a hypercoagulable state (e.g., oral contraceptives, late pregnancy, cancer, inherited thrombophilic disorders, advanced age, venous varicosities, phlebosclerosis).

Diseases and conditions associated with thrombosis and the risk of developing thrombosis or hypercoagulation include, without limitation, acute venous thrombosis, pulmonary embolism, thrombosis during pregnancy, hemorrhagic skin necrosis, acute or chronic disseminated intravascular coagulation (DIC), clot formation from surgery, long bed rest, long periods of immobilization, conditions that preclude or restrict movement such as partial or complete paralysis, morbid obesity, disorders that impede oxygen uptake and absorption such as lung disorders including lung cancer, chronic obstructive pulmonary disease (COPD), emphysema, drug related fibrosis, cystic fibrosis, venous thrombosis, fulminant meningococcemia, acute thrombotic stroke, acute coronary occlusion, acute peripheral arterial occlusion, massive pulmonary embolism, axillary vein thrombosis, massive iliofemoral vein thrombosis, occluded arterial cannulae, occluded venous cannulae, cardiomyopathy, venoocclusive disease of the liver, hypotension, decreased cardiac output, decreased vascular resistance, pulmonary hypertension, diminished lung compliance, leukopenia, inflammatory diseases, metabolic syndrome, and thrombocytopenia.

The term “hemostasis” refers to a coordinated mechanism that maintains the integrity of blood circulation following injury to the vascular system. In normal circulation without vascular injury, platelets are not activated and freely circulate. Vascular injury exposes sub-endothelial tissue to which platelets can adhere. Adherent platelets will attract other circulating platelets to form a preliminary plug that is particularly useful in closing a leak in a capillary or other small vessel. These events are termed primary hemostasis. This is, typically, rapidly followed by secondary hemostasis that involves a cascade of linked enzymatic reactions that result in plasma coagulation to reinforce the primary platelet plug. Later, as wound healing occurs, the platelet aggregate and fibrin clot are degraded as wound healing ensues. In accordance with the present invention, hemostasis is not compromised when the function of one or more of platelet activation, protease-activated receptors, FXIII, protein C or thrombin activatable fibrinolysis inhibitor is retained. The term “not compromised,” as used herein with respect to hemostasis, refers to less than a 20% decrease (e.g., less than about 20%, 19%, 18%, 17%, 16%, 15%, 14%, 13%, 12%, 11%, 10%, 9%, 8%, 7%, 6%, 5%, 4%, 3%, 2%, or 1% decrease) in platelet activation, protease-activated receptors, FXIII, protein C or thrombin activatable fibrinolysis inhibitor function or activity.

The term “coagulation” refers to the process of polymerization of fibrin monomers, resulting in the transformation of blood or plasma from a liquid to a gel phase. Coagulation involves a series of zymogen activation reactions. At each stage, a precursor protein or zymogen, is converted to an active protease by cleavage of one or more peptide bonds in the precursor molecule. The components that can be involved at each stage include a protease from the preceding stage, a zymogen, a non-enzymatic protein cofactor, calcium ions, and an organizing surface that is provided by the damaged blood vessel and platelets in vivo. The final protease to be generated is thrombin (factor IIa).

Fibrinogen is a 330,000 dalton protein composed of three pairs of polypeptide chains (designated α, β and γ) covalently linked by disulfide bonds. Thrombin converts fibrinogen to fibrin monomers (Factor IA) by cleaving fibrinopeptide A (FpA, 16 amino acid residues) and B (FpB, 14 amino acid residues) from the amino-terminal ends of the a and i chains respectively. Removal of the fibrinopeptide allows the fibrin monomers to form a gel. Initially, the fibrin monomers are bound to each other non-covalently. Subsequently, factor XIIIa catalyzes an interchain transglutamination reaction that cross-links adjacent fibrin monomers to enhance the strength of the clot, Fibrin is a substrate of FXIII and fibrinogen/fibrin bind FXIII, however, fibrinogen is not needed to activate of FXIII..

The protease zymogens involved in coagulation include factors II (prothrombin), VII, IX, X, XI, XII, and prekallikrein. Factors V and VIII are homologous 350,000 dalton proteins. Factor VIII circulates in plasma bound to von Willebrand factor, while factor V is present both free in plasma and as a component of platelets. Thrombin cleaves V and VIII to yield activated factors (Va and VIIIa) that have at least 50 times the coagulant activity of the precursor forms. Factors Va and VIIIa have no enzymatic activity themselves, but serve as cofactors that increase the proteolytic efficiency of Xa and IXa, respectively. Tissue factor (TF) is a non-enzymatic lipoprotein cofactor that greatly increases the proteolytic efficiency of VIIa. It is present on the surface of cells that are not normally in contact with blood and plasma (e.g. fibroblasts and smooth muscle cells) since they are abluminal to the endothelium. TF is a key factor that initiates coagulation outside a broken blood vessel.

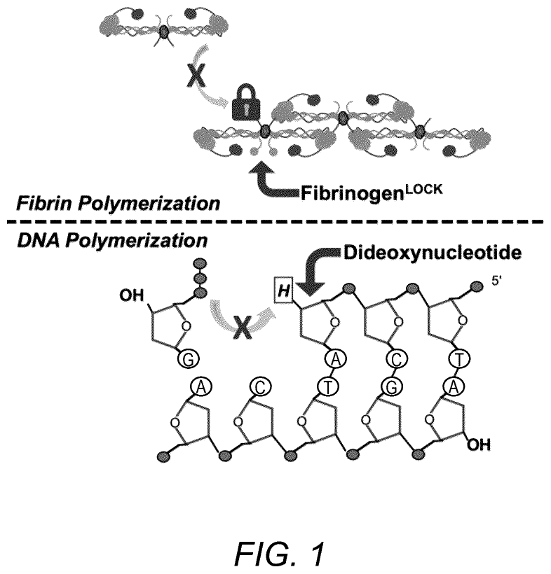

A “nonpolymerizable fibrinogen” refers to fibrinogen Aa (Fga) chain that has been selectively mutated to eliminate thrombin-mediated removal of FpA. The wild-type human fibrinogen alpha chain precursor is a 644 amino acid residue polypeptide. See, e.g., GENBANK Accession No. NP_068567. Removal of the 19 amino acid residue signal peptide results in a 625 amino acid residue fibrinogen polypeptide (SEQ ID NO:3). Thrombin cleavage of wild-type human fibrinogen occurs at the Arg16-Gly17 peptide bond of fibrinogen Aa (SEQ ID NO:3) thereby releasing fibrinopeptide A and exposing a polymerization site (residues 17-20) on the Aa chain. In some aspects, amino acid residues of Fga are altered that render the Fga sequence insensitive to thrombin but the γ-chain binding pocket available to integrate into growing fibrils is retained. Thus, the nonpolymerizable fibrinogen may act as a ‘fibrin(ogen) lock’ by limiting fibrin formation through ‘capping’ and terminating extension of a growing fibril in much the same way as a dideoxynucleotide terminates DNA polymerization (FIG. 1). In some aspects, the nonpolymerizable fibrinogen has a mutation in the thrombin cleavage site E^{P6}GGGVR^{P1} (SEQ ID NO:1). As is conventional in the art, amino acid residues in a substrate undergoing cleavage are designated P1, P2, P3, P4, etc. in the N-terminal direction from the cleaved bond. Cleavage occurs between P1 and P1′ with P1 being composed of the amino acid arginine at position 16 in the Fga molecule (SEQ ID NO:3) and P1′ designating the amino acid glycine at position 17 in the Fga molecule (SEQ ID NO:3). In some aspects, the nonpolymerizable fibrinogen has a mutation in the thrombin cleavage site of SEQ ID NO:1, wherein the mutation is a E^{P6}GGGVR^{P1} (SEQ ID NO:1) to A^{P6}DDDDK^{P1} (SEQ ID NO:2) mutation. In some aspects, the nonpolymerizable fibrinogen has an amino acid sequence identity of at least 80%, 85%, 90%, 95%, 96%, 97%, 98%, or 99% with the amino acid sequence of SEQ ID NO:3, has the E^{P6}GGGVR^{P1} (SEQ ID NO:1) to A^{P6}DDDDK^{P1} (SEQ ID NO:2) mutation and retains the ability to integrate into growing fibrils. In some aspects, the nonpolymerizable fibrinogen has an amino acid sequence identity of at least 80%, 85%, 90%, 95%, 96%, 97%, 98%, or 99% with the amino acid sequence of SEQ ID NO:8 and retains the ability to integrate into growing fibrils. Nonpolymerizable fibrinogen may be produced by conventional recombinant protein expression, chemical synthesis, or a combination thereof.

As used herein, the term “polypeptide” or “protein” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

By an “isolated” polypeptide is intended a polypeptide that is not in its natural milieu. No particular level of purification is required. For example, an isolated polypeptide can be removed from its native or natural environment. Recombinantly produced polypeptides and proteins expressed in host cells are considered isolated for purpose of the invention, as are native or recombinant polypeptides that have been separated, fractionated, or partially or substantially purified by any suitable technique.

“Antithrombotics” or “antithrombotic agents” include agents that inhibit platelet aggregation (antiplatelet agents), inhibit formation of fibrin strands (anticoagulants), and/or dissolve existing clots (fibrinolytics). Antiplatelet agents and anticoagulants target each component of clot formation and can be used to prevent thrombogenesis, but have no effect on existing clots, other than limiting their progression. By comparison, fibrinolytic (thrombolytic) agents can dissolve an existing thrombus.

A “required dose” of an agent is a measured amount of the agent that, when administered alone, has a therapeutic effect.

The phrase “complementing the effect of” an agent refers to the beneficial effect of a second component on the effect of a first component. In some embodiments, the effect of the second component is different from the effect of the first component. It some embodiments, the effect of the second component is the same as the effect of the first component. In some embodiments, the effect of the second component may supplement the effect of the first ingredient, i.e., the second component produces an additive effect or synergistically enhances the medicinal effect of the first component.

The present invention is based upon the finding that a nonpolymerizable fibrinogen variant in the heterozygous state (Fga^{WT/EK}) or the homozygous state (Fga^{EK/EK}) provides protection from venous and arterial thrombosis, while preserving hemostatic potential linked to normal platelet function. In particular, it was shown that the presence of nonpolymerizable fibrinogen in the milieu with normal fibrinogen suppresses fibrin matrix formation leading to a significant reduction in thrombosis incidence and thrombus size without directly impeding thrombin-mediated activation of platelets, protease-activated receptors, FXIII, protein C or thrombin activatable fibrinolysis inhibitor. In addition, in vitro turbidity assays mixing platelet-poor plasma from Fga^{EK/EK} and Fga^{WT/EK} mice demonstrated that introduction of nonpolymerizable fibrinogen significantly reduces fibrin formation in a dose-dependent manner. Thus, nonpolymerizable fibrinogen finds use in methods for inhibiting or reducing the risk of venous or arterial thrombosis without compromising hemostasis, reducing the number and/or size of thrombi formed in a subject without compromising hemostasis, and reducing a required dose or complementing the effect of an antithrombotic agent in the treatment of thrombosis.

The nonpolymerizable fibrinogen of the invention may be administered to any subject in which inhibition of venous or arterial thrombosis or risk of developing a venous or arterial thrombosis would be beneficial. In particular, it is contemplated that the nonpolymerizable fibrinogen of the invention is particularly useful as an antithrombotic agent that does not compromise hemostasis, and as such is a superior alternative to traditional antithrombotic agents. For example, in the coronary arteries, occlusive thrombus formation often follows the rupture of atherosclerotic plaque. This occlusion is the major cause of acute myocardial infarction and unstable angina. Coronary occlusions can also occur following infections, inflammation, thrombolytic therapy, angioplasty, and graft placements. Similar principles apply to other parts of the arterial vasculature and include, among others, thrombus formation in the carotid arteries, which is the major cause of transient or permanent cerebral ischemia and stroke.

Venous thrombosis often follows stasis, infections, inflammatory reactions, and major surgery of the lower extremities or the abdominal area. Deep vein thrombosis results in reduced blood flow from the area distal to the thrombus and predisposes to pulmonary embolism. Pulmonary embolism is a major cause of post-surgical mortality. Disseminated intravascular coagulation (DIC) and acute respiratory distress syndrome (ARDS) where the nonpolymerizable fibrinogen is useful commonly occur within all vascular systems during bacterial sepsis, entry of foreign material into the blood stream following, e.g., trauma and child birth, immune reactions, inflammation, certain viral infections, certain platelet disorders, and cancer. Disseminated intravascular coagulation is a severe complication of many disease conditions and some drug treatments, including, for example, heparin. Thrombotic consumption of coagulation factors and platelets, and systemic coagulation results in the formation of life-threatening thrombi occurring throughout the microvasculature leading to local or widespread hypoxia and organ failure.

Thus, in some embodiments, a method is provided for inhibiting or reducing the risk of developing thrombosis in a subject in need thereof (e.g., a subject at increased risk of developing thrombosis) comprising administering to the subject an effective amount of nonpolymerizable fibrinogen, particularly where the thrombosis is associated with, e.g., follows or is the cause of: (1) acute coronary syndromes such as myocardial infarction, unstable angina, refractory angina, occlusive coronary thrombus occurring post-thrombolytic therapy or post-coronary angioplasty; (2) ischemic cerebrovascular syndromes including embolic stroke, thrombotic stroke, or transient ischemic attacks; (3) thrombosis occurring in the venous system occurring either spontaneously or in the setting of malignancy, trauma, or surgery, including pulmonary thromboembolism; (4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin-induced thrombocytopenia; (5) thrombotic complications associated with extracorporeal circulation (e.g., renal dialysis, cardiopulmonary bypass or other oxygenation procedure such as ECMO treatment, and plasmaphoresis); (6) thrombotic complications associated with instrumentation (e.g., cardiac or other intravascular catheterization, intraaortic balloon pump, and coronary stent or cardiac valve); and/or (7) complications associated with fitting of prosthetic devices. In some embodiments, the venous or arterial thrombosis is associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts, and/or prostheses. In other embodiments, the subject is receiving ECMO treatment and/or surgery. In still other embodiments, the subject has a condition associated with increased risk of thrombosis. In some embodiments, the thrombosis is a large vascular thrombosis, a small vascular thrombosis or a microvascular thrombosis. In further embodiments, the subject is human. In other embodiments, the nonpolymerizable fibrinogen comprises a mutation in the thrombin cleavage site, optionally wherein the mutation comprises a E^{P6}GGGVR^{P1} (SEQ ID NO:1) to A^{P6}DDDDK^{P1}(SEQ ID NO:2) mutation.

As demonstrated herein, the introduction of nonpolymerizable fibrinogen in the milieu with normal fibrinogen suppresses fibrin matrix formation leading to a significant reduction in thrombosis incidence and thrombus size without directly impeding thrombin-mediated activation of platelets, protease-activated receptors, FXIII, protein C or thrombin activatable fibrinolysis inhibitor. Thus, in some embodiments, a method is provided for reducing the number and/or size of thrombi formed in a subject without compromising hemostasis, comprising administering to a subject in need thereof an effective amount of nonpolymerizable fibrinogen. In some embodiments, administration of nonpolymerizable fibrinogen provides at least about a 1% (e.g., about 2%, 3%, 4%, 5%, 6%, 7%, 8%, 9%, 10%, 15%, 20%, 25%, 30%, 35%, 40%, 45%, 50%, 55%, 60%, 65% or 70%) reduction in number of thrombi and/or at least about a 1 mg (e.g., about 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg) reduction in the mass of individual thrombi as compared to a subject with the same or similar condition not receiving treatment with nonpolymerizable fibrinogen. In some embodiments, the reduced number and/or size of thrombi formed exhibit a heterogenous distribution of platelets and neutrophils. In some embodiments, the thrombi are associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts, and/or prostheses. In other embodiments, the subject is receiving ECMO treatment and/or surgery. In still other embodiments, the subject has a condition associated with increased risk of thrombosis. In further embodiments, the subject is human. In other embodiments, the nonpolymerizable fibrinogen comprises a mutation in the thrombin cleavage site, optionally wherein the mutation comprises a E^{P6}GGGVR^{P1} (SEQ ID NO:1) to A^{P6}DDDDK^{P1}(SEQ ID NO:2) mutation.

Therapeutic efficacy of the nonpolymerizable fibrinogen in inhibiting thrombosis or reducing the number and/or size of thrombi may be assessed by conventional functional and/or physiological measurements. Exemplary physiological parameters include blood pressure or flow (velocity) inside the vasculature. The pressure and flow measurements may be compared to a reference baseline. The reference baseline may be measurements associated with a normal, healthy population or a reference baseline specific to the individual subject to therapy (e.g., measured pressure/flow levels of the patient prior to the thrombosis). Other assessment indicators include, for example, endovascular echo changes or vascular wall thickness comparisons. For example, vascular wall thickness and intraluminal echo can be assessed by gray-scale sonography, while iliac, femoral vein blood flow and femoral vein valve insufficiency may be evaluated with Doppler ultrasound.

Assessment of hemostasis can be carried out using any standard template skin bleeding time test (e.g. Surgicutt®, International Technidye Corp). Experimentally, this and similar tests (e.g., Simplate bleeding times) have been shown to be sensitive to the effects of therapeutic anticoagulants, anti-platelet agents, and coagulation abnormalities in humans and non-human primates (Gruber et al., (2007) Blood 109:3733-3740; Smith et al. (1985) Am. J. Clin. Pathol, 83:211-215; Payne et al. (2002) J. Vasc. Surg. 35:1204-1209). For indirect assessment of hemostasis, prothrombin time (PT) and activated partial thromboplastin time (aPTT) measurements may be used.

In some aspects, the nonpolymerizable fibrinogen of the methods of the invention is administered in combination with an antithrombotic agent and/or anticoagulant agent. Any such conventional agent may be used including, but not limited to, a direct or indirect thrombin inhibitor, a Factor X inhibitor, a Factor IX inhibitor, a Factor XII inhibitor, a Factor V inhibitor, a Factor VIII inhibitor, a Factor XIII inhibitor, a Factor VII inhibitor, a tissue factor inhibitor, a profibrinolytic agent, a fibrinolytic or fibrinogenolytic agent, a carboxypeptidase B inhibitor, a platelet inhibitor, a selective platelet count reducing agent, or a Factor XI inhibitor.

Direct thrombin inhibitors include, e.g., argatroban and derivatives or analogs thereof, hirudin and recombinant or synthetic derivatives or analogs thereof, derivatives of the tripeptide Phe-Pro-Arg, chloromethylketone derivatives, lepirudin, bivalirudin, dabigatran, ximelagatran and derivatives, metabolites, or analogs thereof, anion binding exosite inhibitors, and RNA/DNA aptamers.

Indirect thrombin inhibitors include, e.g., heparin, enoxaparin, warfarin and other coumarin derivatives, dermatan, and thrombomodulin.

Factor X inhibitors include, e.g., direct factor Xa inhibitors, rivaroxaban, apixaban, antibodies to factor X, inactivated factor Xa, or analogs and derivatives thereof.

Factor IX inhibitors include, e.g., antibodies to factor IX, direct factor IXa inhibitors, or inactivated factor IXa, or analogs and derivatives thereof.

Factor XII inhibitors include, e.g., direct factor XII inhibitors, corn trypsin inhibitor, antibodies to FXII, antisense oligonucleotides, or inactivated factor XIIa or analogs and derivatives thereof.

Factor V inhibitors include, e.g., antibodies to factor V, activated protein C, protein S, or analogs and derivatives thereof.

Factor VIII inhibitors include, e.g., antibodies to FVIII, activated protein C, protein S, or analogs and derivatives thereof.

Factor XIII inhibitors include, e.g., antibodies to factor XIII, direct factor XIIIa inhibitors, or inactivated factor XIIIa.

Factor VII inhibitors include, e.g., antibodies to factor VII, tissue factor pathway inhibitor, inactivated factor VIIa, or direct factor VIIa inhibitor or analogs and derivatives thereof.

Tissue factor inhibitors include, e.g., tissue factor pathway inhibitor, antibodies to tissue factor, or analogs and derivatives thereof.

Profibrinolytic agents include, e.g., urokinase, streptokinase, tissue plasminogen activator or derivatives thereof.

Fibrinolytic or fibrinogenolytic agents include, e.g., plasmin, microplasmin, ancrod, or derivatives thereof.

Platelet inhibitors include, e.g., aspirin, cangrelor, cilostazol, clopidogrel, dipyridanole, prasugrel, ticlopidine, ticagrelor, vorapaxar, abeiximab, eptifibatide, tirofiban or derivatives thereof.

Selective platelet count reducing agents include, e.g., hydroxyurea, anagrelide, or derivatives thereof.

Factor XI inhibitors include, e.g., direct factor XIa inhibitors, antibodies to factor XI, antisense oligonucleotides, inactivated factor XIa, or analogs and derivatives thereof.

Conventional antithrombotic agents, such as those described herein, may be dangerous or even fatal when administered at their maximally effective doses. Accordingly, in another aspect of this invention, a method is provided for reducing a required dose or complementing the effect of an antithrombotic agent in the treatment of thrombosis in a subject in need thereof comprising administering to the subject an effective amount of the nonpolymerizable fibrinogen in combination with the antithrombotic agent. Administration of the nonpolymerizable fibrinogen in combination with the antithrombotic agent may avoid or provide a reduction in one or more of the side effects associated with administration of higher doses of the anticoagulant agent. In some embodiments, nonpolymerizable fibrinogen complements the effect of the antithrombotic agent when the antithrombotic agent is administered at a known, conventional dose. See, e.g., Table 1. In other embodiments, the antithrombotic agent is administered at a reduced dose that is about 10% (e.g., about 20%, 25%, 30%, 35%, 40%, 45%, 50%, 55%, 60%, 65%, 70%, 80%, 90% or 95%) of the known, conventional dose.

TABLE 1
Conventional doses of antithrombotic agent.

| Antithrombotic Agent | Recommended Daily Dose | |
|---|---|---|
| Aspirin | 50-150 | mg |
| Clopidogrel | 75 | mg |
| Rivaroxaban | 15-20 | mg |
| Dabigatran | 110-150 | mg |
| Apixaban | 5 | mg |
| Low Molecular Weight Heparin | 120-225 | mg |
| Enoxaparin | 1 | mg/kg |
| Warfarin (Coumadin) | 2-5 | mg |
| Lepirudin | 0.4 | mg/kg |
| Bivalirudin | 1 | mg/kg |
| Abciximab | 0.25 | mg/kg |
| Eptifibatide | 180 | μg/kg |

The nonpolymerizable fibrinogen of the invention may be formulated according to known methods for preparing pharmaceutically useful compositions, such as by admixture with a pharmaceutically acceptable carrier. Suitable carriers and their formulations are described, for example, in Remington's Pharmaceutical Science; 21^{st} ed. 2005. In order to form a pharmaceutically acceptable composition suitable for effective administration, such compositions will contain an effective amount of the nonpolymerizable fibrinogen either alone, or in combination with an antithrombotic agent, anticoagulant, and/or a suitable amount of carrier. In some embodiments, the invention provides a pharmaceutical composition comprising an effective amount of a nonpolymerizable fibrinogen and an antithrombotic agent in admixture with a pharmaceutically acceptable carrier.

Pharmaceutical compositions may be administered in admixture with a suitable carrier, diluent, or excipient such as sterile water, physiological saline, glucose, or the like. The compositions may contain auxiliary substances such as wetting or emulsifying agents, pH buffering agents, adjuvants, gelling or viscosity enhancing additives, preservatives, flavoring agents, colors, and the like, depending upon the route of administration and the preparation desired.

Pharmaceutical compositions may be formulated for immediate release or controlled release. The “absorption pool” represents a solution of the drug administered at a particular absorption site, and kᵣ, kₐ, and kₑ are first-order rate constants for: (1) release of the drug from the formulation; (2) absorption; and (3) elimination, respectively. For immediate release dosage forms, the rate constant for drug release kᵣ is far greater than the absorption rate constant kₐ. For controlled release formulations, the opposite is true, i.e., kᵣ<kₐ, such that the rate of release of drug from the dosage form is the rate-limiting step in the delivery of the drug to the target area. The term “controlled release” as used herein includes any nonimmediate release formulation, including but not limited to sustained release, delayed release and pulsatile release formulations.

Pharmaceutical compositions comprising nonpolymerizable fibrinogen of the invention may be administered in dosages and by techniques well known to those skilled in the medical or veterinary arts, taking into consideration such factors as the age, sex, weight, species and condition of the particular subject, and the route of administration. The route of administration can be via any route that delivers a safe and effective dose of a nonpolymerizable fibrinogen of the invention to the blood of an animal or human. Forms of administration, include, but are not limited to, systemic, enteral, parenteral, and topical routes of administration. Enteral routes include oral and gastrointestinal administration. Parenteral routes include intravenous, intraarterial, intramuscular, intraperitoneal, subcutaneous, transdermal, and transmucosal administration. Other routes of administration include epidural or intrathecal administration. In some embodiments, topical administration may be performed by applying a nonpolymerizable fibrinogen-containing dressing and/or catheter to the thrombus area. In some embodiments, the nonpolymerizable fibrinogen may be administered before, during and/or after other therapies. For example, when used in combination with surgery, nonpolymerizable fibrinogen may be administered before surgery (e.g., 1, 2, 3, 4, 5, or 6 hours) and/or after surgery (e.g., 1, 2, 3, 4, 5, 6, or 7 days, weeks or months).

The effective dosage and route of administration are determined by the therapeutic range, whether the nonpolymerizable fibrinogen is used alone or in combination with another agent, and by known factors, such as the age, weight, and condition of the subject, as well as LD₅₀ and other screening procedures that are known and do not require undue experimentation.

The term “dosage” as used herein refers to the amount of a nonpolymerizable fibrinogen of the invention administered to an animal or human. The nonpolymerizable fibrinogen may be delivered to the recipient as a bolus or by a sustained (continuous or intermittent) delivery. When the delivery of a dosage is sustained over a period, which may be in the order of a few minutes to several days, weeks or months, or chronically for a period of years, the dosage may be expressed as weight of the therapeutic agent/kg body weight of the patient/unit time of delivery.

In some embodiments of the invention, an effective dose of the nonpolymerizable fibrinogen is in the range from about 0.01 mg/kg to about 10 mg/kg, from about 0.1 mg/kg to about 10 mg/kg, from about 1 mg/kg to about 10 mg/kg, from about 1 mg/kg to about 5 mg/kg, from about 0.01 mg/kg to about 1 mg/kg, or from about 0.1 mg/kg to about 1 mg/kg. In other embodiments, the effective dose of nonpolymerizable fibrinogen is about 0.01 mg/kg, about 0.03 mg/kg, about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 5 mg/kg, about 7 mg/kg, about 10 mg/kg, or other such doses falling within the range of about 0.01 mg/kg to about 10 mg/kg. It is recognized that the methods of this invention may comprise a single administration of an effective dose or multiple administrations of an effective dose of the nonpolymerizable fibrinogen.

In embodiments pertaining to a combination therapy comprising an effective amount of nonpolymerizable fibrinogen and an antithrombotic/anticoagulant agent, the combination therapy may be carried out by administration of the different active agents in a single composition, by concurrent administration of the different active agents in different compositions, or by sequential administration of the different active agents. Alternatively or in addition to the use of an antithrombotic/anticoagulant agent, nonpolymerizable fibrinogen may be administered in combination with other drugs including, for example, cardiovascular disease therapeutic drugs, arrhythmia therapeutic drugs, diabetes therapeutic drugs, etc.

To facilitate administration and use in accordance with one or more of the methods herein, the present invention also provides nonpolymerizable fibrinogen and optionally an antithrombotic/anticoagulant agent in an article of manufacture or a kit. The invention also relates to a method for preparing a pharmaceutical composition, an article of manufacture or a kit for inhibiting or reducing the risk of thrombosis or reducing the size or number of thrombi in a subject, which comprises preparing nonpolymerizable fibrinogen, a pharmaceutically acceptable carrier, and optionally an antithrombotic/anticoagulant agent together into the pharmaceutical composition, article of manufacture or kit.

Having described the present invention, the same will be explained in greater detail in the following examples, which are included herein for illustration purposes only, and which are not intended to be limiting to the invention.

### Example 1: Material and Methods

Mice. Animal experiments and protocols were approved by the Animal Care and Use Committee of the University of North Carolina at Chapel Hill. Fga^{EK} and Fga^{-/-} mice were previously described (Prasad et al. (2015) Blood 126(17):2047-2058; Prasad et al. (2015) Blood 126(17):2047-2058). Briefly, the Fga^{EK} targeting vector was generated by PCR-based mutagenesis in which 11 nucleic acid substitutions were introduced into exon 2 of the fibrinogen α-chain gene that converted Fga residues immediately upstream of the thrombin cleavage site from E^{P6}GGGVR^{P1} (SEQ ID NO:1) to A^{P6}DDDDK^{P1} (SEQ ID NO:2) and electroporated into E14Tg2a ES cells. Homologous recombinants were identified by PCR analysis using primers complementary to HPRT (5′-CATTGCTCAGCGGTGCTGTCCATCTGCACG-3′; SEQ ID NO:4) and the Fga gene (5′-TTCCGGTACCCCAGTCTCCAGTGCC-3′; SEQ ID NO:5). The mutation was confirmed by PCR analysis using primers upstream (5′-AGTAGTTAACAGGCCATAGGTGAATT-3′; SEQ ID NO:6) and downstream(5′-AAGCCAGCAGTACTGGCTCCTAATGT-3′; SEQ ID NO:7) of the mutated sequence in conjunction with a diagnostic PvuII restriction enzyme digest. Mice carrying the mutant allele were crossed to CMV-Cre mice for removal of the HPRT minigene, and the resultant Fga^{EK} progeny mice that expression the fibrinogen^{LOCK} protein were backcrossed 6 generations to C57Bl/6J (Jackson Labs). Male and female mice between the ages of 8 and 12 weeks were used for experimental procedures.

Mouse models of arterial and venous thrombosis. The inferior vena cava (IVC) stasis model was performed as described (Mwiza et al. (2022) Blood 139(21):3194-3203). The stasis model has near 100% penetrance and provides reproducible thrombi for proteomic and histological analyses. Side branches were ligated, and the lumbar branches cauterized. After 24 hours, thrombi were isolated from the IVC and weighed. Histological analyses were performed on 5 μm sections of formalin-fixed paraffin-embedded tissues using antibodies against fibrinogen (GAM-FGB-BIO, ThermoFisher), CD41 (EPR17876, Abcam) and Ly6G (InVivoMab, Bio X Cell).

The stasis model is performed as described (Aleman et al. (2014) J. Clin. Invest. 124(8):3590-600). The stenosis model maintains a degree of flow, is sensitive to changes in leukocyte tissue factor (TF) activity and provides an additional readout of thrombosis incidence. Complete blood cell counts (CBCs), fibrinogen, thrombin-antithrombin (TAT), plasmin-α2-antiplasmin (PAP), and fibrin degradation products (FDPs) may be measured pre- and post-thrombotic challenge. Mice are euthanized 24 or 48 hours after surgery for detection of thrombus formation. Thrombi are then separated from the vessel wall, weighed, and frozen or fixed for analysis. Histological analyses of thrombi include H&E staining to quantify thrombus organization and composition (i.e., RBC-to-platelet ratio and leukocyte content) using ImageJ (at 5 different tissue depths and 5 sections/depth/thrombus). Thrombi sections are also stained for RBC (anti-TER-119/anti-glycophorin A), platelets (anti-CD41/CD42), neutrophils (anti-Ly6C), and macrophages (anti-F4/80) with positive cells quantified per area, as well as fibrin (anti-fibrinogen antibody) and fibrinolytic pathway proteins (tPA, uPA, PAI-1, and α2-antiplasmin), as described (Aleman et al. (2014) J. Clin. Invest. 124(8):3590-600; Singh et al. (2020) Front. Cardiovas. Med. 7:608899). In parallel experiments, total protein preparations of thrombi are analyzed for fibrin(ogen), platelet (anti-CD41), and RBC (anti-TER-119) content.

The ferric chloride (FeCl₃) model of arterial thrombosis was performed as described (Lee et al. (2022) J. Thromb. Haemost. 20(2):422-433; Kawano et al. (2023) Thromb. Haemost. 123(5):501-509). Briefly, filter paper (1-2 mm²) soaked in 4-10% FeCl₃ solution was placed on the carotid artery for 1-3 minutes. Blood flow was measured for 30 minutes using a microvascular ultrasonic flow probe. Time to occlusion was defined as cessation of blood flow for Q 2 minutes. An embolic event was defined as change in blood flow of >0.5 mL/minute within 1 minute. Intravital microscopy was performed by injecting ALEXA FLUOR®488 dye-conjugated antibodies to GPIX (2.5 □g, clone Xia.B4, Emfret Analytics) prior to FeCl₃ application.

Mouse models of hemostasis and wound healing. Tail bleeding times were measured as described (Hur et al. (2022) Blood 139(9):1374-1388). A 3-mm tail tip was excised from anesthetized mice and the tail was submerged in Tris-buffered saline (pH 7.5) containing 2 mM CaCl₂ at 37□C until cessation of bleeding was sustained for over 30 seconds. Laser-induced saphenous vein injury was performed as described (Paul et al. (2020) Arterioscler. Thromb. Vasc. Biol. 40(8):1891-1904). Briefly, mice were injected with ALEXA FLUOR®488 dye-conjugated antibodies to GPIX (2.5 μg, clone Xia.B4, Emfret Analytics) to detect platelets, and labelled fibrinogen (100 μg/mouse) isolated from Fga^{WT/WT}, Fga^{EK/EK} or Fga^{EK/EK} mice. Fga-mice were injected with ALEXA FLUOR®647 dye-conjugated antibodies against fibrin (2 μg/mouse; gift from Rodney Camire). Platelet and fibrin accumulation at laser injury sites were assessed by intravital microscopy. Wound healing assay was performed as described (Bugge et al. (1996) Cell 87(4):709-719). Briefly, a 1-cm incision was made on the dorsal skin and monitored daily for 15 days. The wound site was fixed for histological evaluation.

Statistical analyses. Data was analyzed using Prism 9. Comparisons of multiple groups were performed using one-way ANOVA, Kruskal-Wallis or Mantel-Cox tests. Results were considered significant when p<0.05.

### Example 2: Fga^{EK} Mice are Protected from Arterial Thrombosis

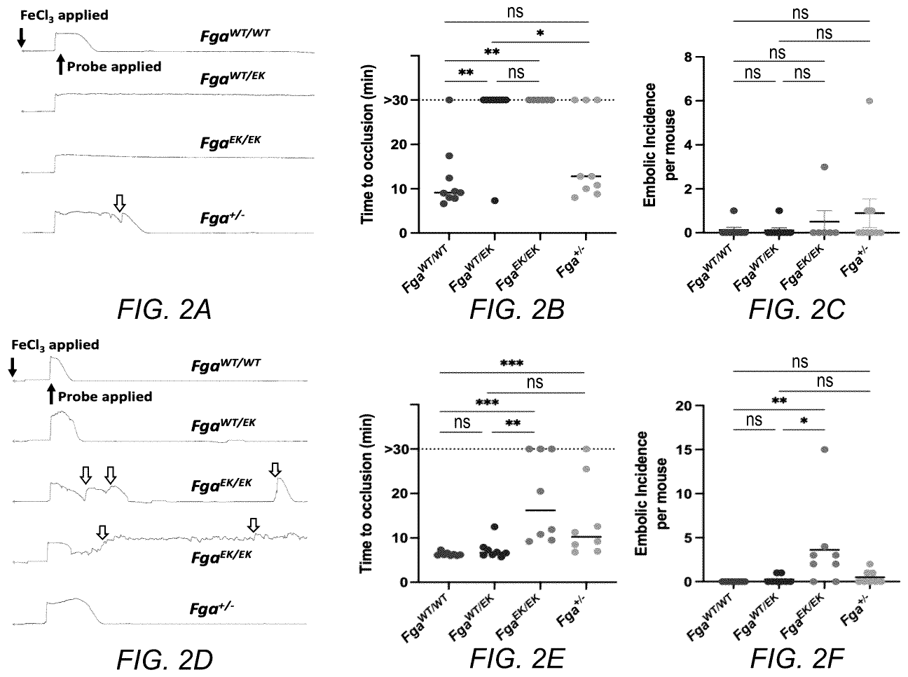

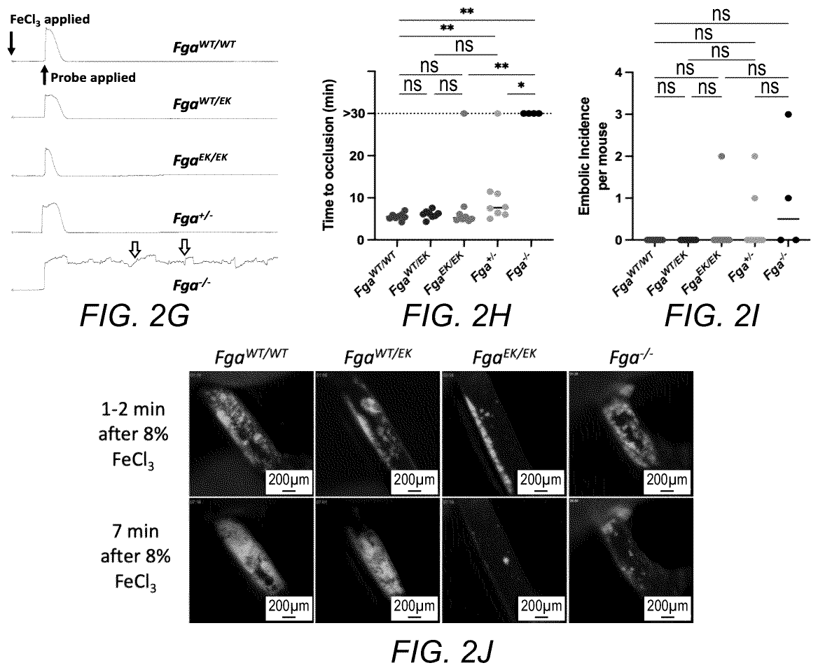

To examine the impact of nonpolymerizable fibrinogen on arterial thrombosis, 3-minute FeCl₃ carotid artery injuries were performed. At 4% FeCl₃ challenge dose, Fga^{EK/EK}mice were significantly protected from vascular occlusion. Interestingly, Fga^{WT/EK} mice also displayed near complete protection with only one animal developing an occlusive thrombus (FIGS. 2A-2B). Only a subset of Fga^{+/-} mice, which express 50% of fibrinogen^{WT} levels, developed occlusive thrombi, without a significant difference in the overall time to occlusion from Fga^{WT} mice (FIGS. 2A-2B). In Fga^{+/-} mice, a trend toward an increased number of embolic events was observed (FIG. 2C). Following a 5% FeCl₃ challenge, Fga^{EK/EK} mice displayed a reduction in the incidence of occlusive thrombi and a significant delay in time to occlusion compared to Fga^{WT/WT} and Fga^{WT/EK} mice (FIGS. 2D-2E). The number of embolic events was significantly elevated in Fga^{EK/EK} mice relative to Fga^{WT/WT} and Fga^{WT/EK} animals (FIG. 2F) at this challenge dose. Increasing to 10% FeCl₃ resulted in Fga^{WT/WT}, Fga^{WT/EK} and Fga^{EK/EK} mice displaying comparable flow profiles characterized by similar mean times to occlusion (FIGS. 2G-2H). Time to occlusion in Fga^{+/-} mice was similar compared to Fga^{WT/EK} mice but delayed compared to Fga^{WT/WT} mice. In contrast, Fga^{-/-} mice displayed little reduction in flow and did not develop occlusive thrombi over the entire 30-minute observation period (FIGS. 2G-2H). Few embolic events were detected in any genotype with the 10% challenge (FIG. 2I). The development of arterial thrombi was visualized by intravital microscopy immediately after challenging the mice with 8% FeCl₃ for only 1 minute. There was a gradual and persistent accumulation of platelets at the injury site in Fga^{WT/WT} and Fga^{WT/EK} mice (FIG. 2J). In contrast, Fga^{EK/EK} platelets formed small thrombi that were quickly dislodged. Results in Fga^{-/-} mice were similar to Fga^{EK/EK} mice, except that larger platelet clusters formed before embolization.

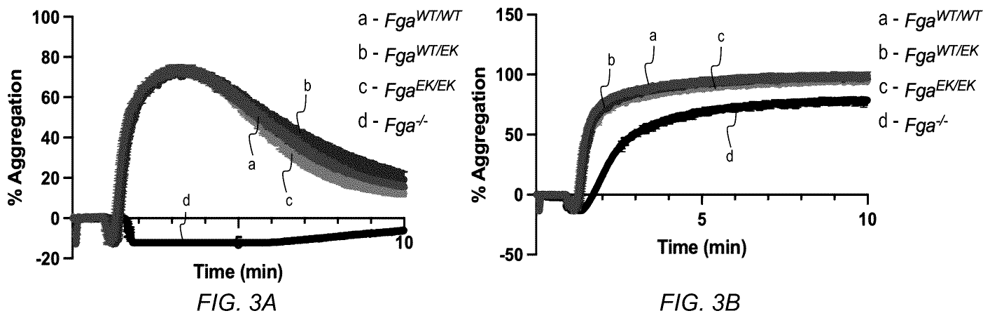

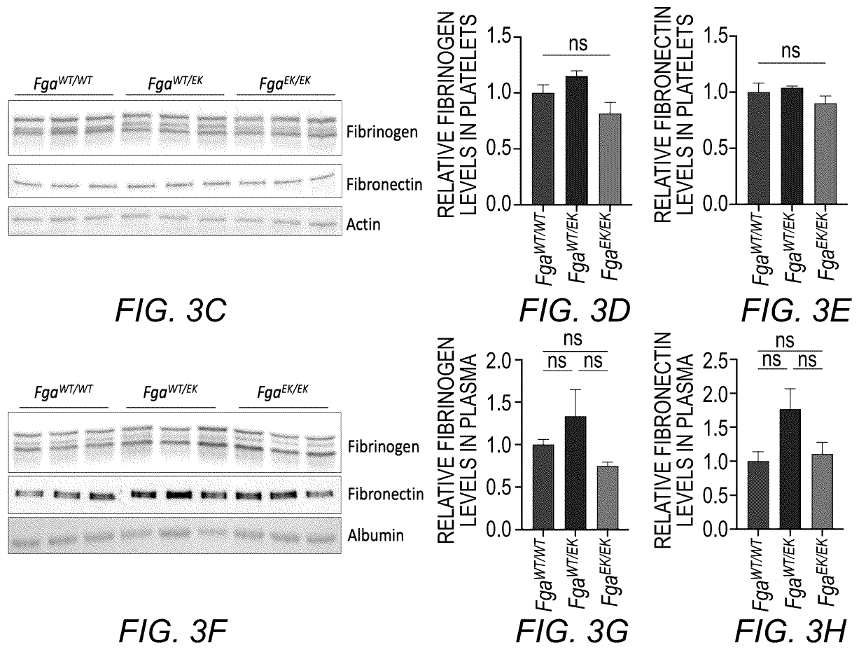

The formation of occlusive thrombi following severe injury in Fga^{EK/EK} mice was hypothesized to be linked to preserved fibrinogen^{EK}-dependent platelet functions. Platelet aggregation responses were measured in platelet-rich plasma isolated from mice of each genotype following activation with ADP or protease activated receptor 4-activating peptide (Par4p). Equivalent aggregation was observed for Fga^{WT/WT} Fga^{WT/EK} and Fga^{EK/EK} platelets regardless of the activator (FIGS. 3A-3B). Fga^{-/-} platelets failed to aggregate in response to ADP (FIG. 3A) but displayed preserved aggregation in response to Par4p stimulation (FIG. 3B), similar to findings in previous studies (Hur et al. (2022) Blood 139(9):1374-1388; Ni et al. (2000) J. Clin. Invest. 106(3):385-392; Yang et al. (2006) J. Thromb. Haemost. 4(10):2230-2237; Reheman et al. (2009) Blood 113(8):1809-1817). The relative amounts of fibrinogen and fibronectin in washed platelets and in plasma were similar among Fga^{WT/WT}, Fga^{WT/EK} and Fga^{EK/EK} mice (FIGS. 3C-3H), indicating that the Fga^{EK} mutation does not alter the uptake or compartmentalization of these key extracellular matrix molecules in platelets.

### Example 3: Fga^{EK/EK} Mice are Protected from Venous Thrombosis

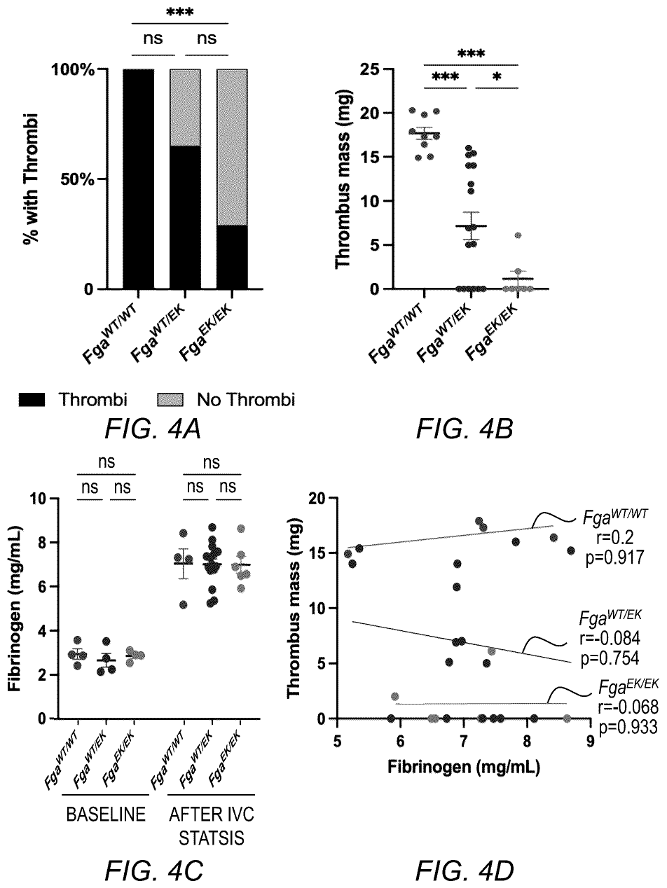

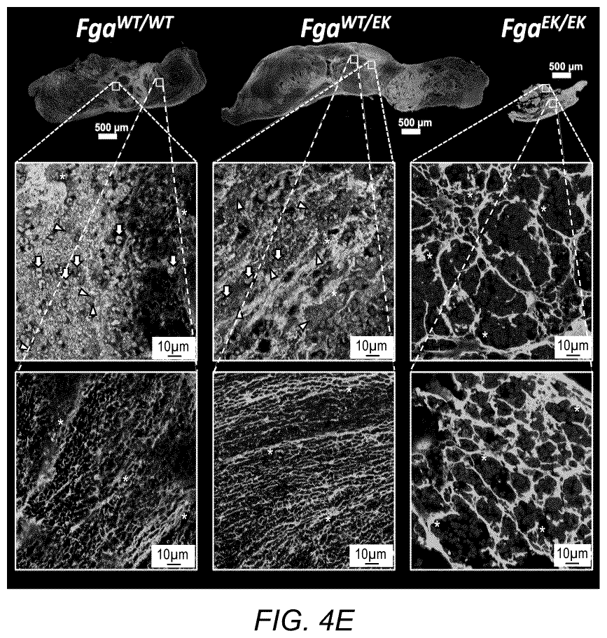

To examine the impact of nonpolymerizable fibrinogen on venous thrombosis, an IVC stasis model was performed. Large thrombi were observed in all Fga^{WT/WT} mice after 24 hours. However, Fga^{EK/EK} mice had significantly reduced incidence of thrombus formation compared to Fga^{WT/WT} mice, with only ˜30% of animals developing measurable thrombi (FIG. 4A). In addition, ˜30% of Fga^{WT/EK} developed no measurable thrombi, although this difference did not achieve statistical significance relative to Fga^{WT/WT} mice (FIG. 4A). Moreover, the masses of thrombi that did form were reduced in both Fga^{WT/EK} and Fga^{EK/EK} mice relative to Fga^{WT/WT} mice (FIG. 4B). Previous work revealed that the IVC stasis surgery drives an acute phase response resulting in significantly elevated plasma fibrinogen levels (Hur et al. (2022) Blood 139(9):1374-1388). Comparable increases in fibrinogen levels were observed in each of the genotypes following the IVC stasis procedure (FIG. 4C) and no correlations were observed between thrombus mass and the circulating fibrinogen levels after IVC stasis (FIG. 4D). Fibrin networks were observed in Fga^{WT/WT} and Fga^{WT/EK} thrombi, while Fga^{EK/EK} thrombi contained thick aggregates or clumps of fibrinogen that were morphologically distinct (FIG. 4E). In Fga^{WT/WT} and Fga^{WT/EK} thrombi, there was heterogenous distributions of platelets and neutrophils within each thrombus. However, the material isolated from those few Fga^{EK/EK} mice with thrombi consisted of areas that contained neutrophils and RBCs but were largely devoid of platelets (FIG. 4E).

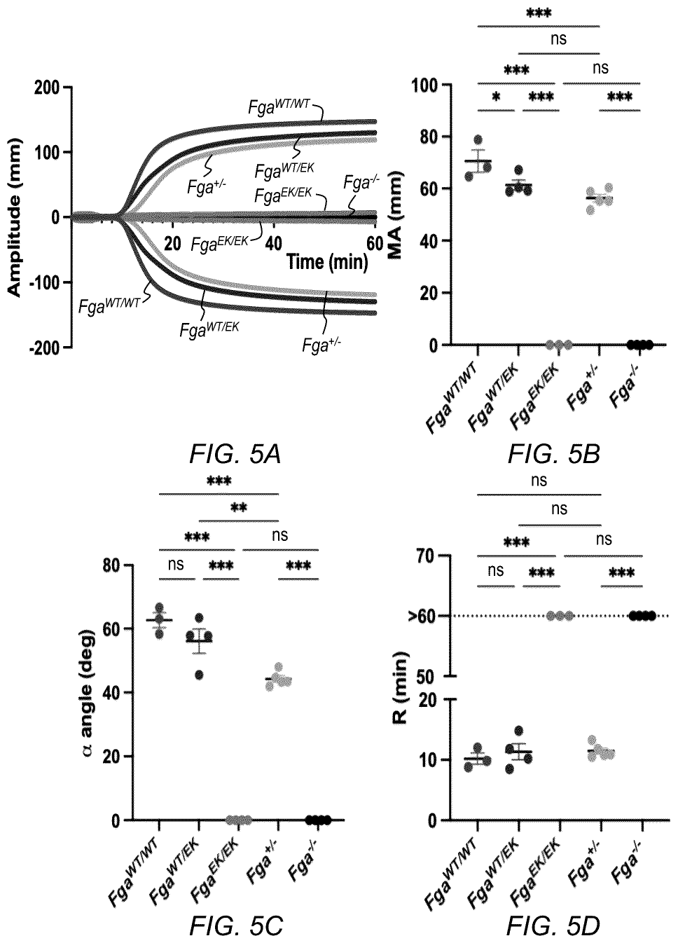

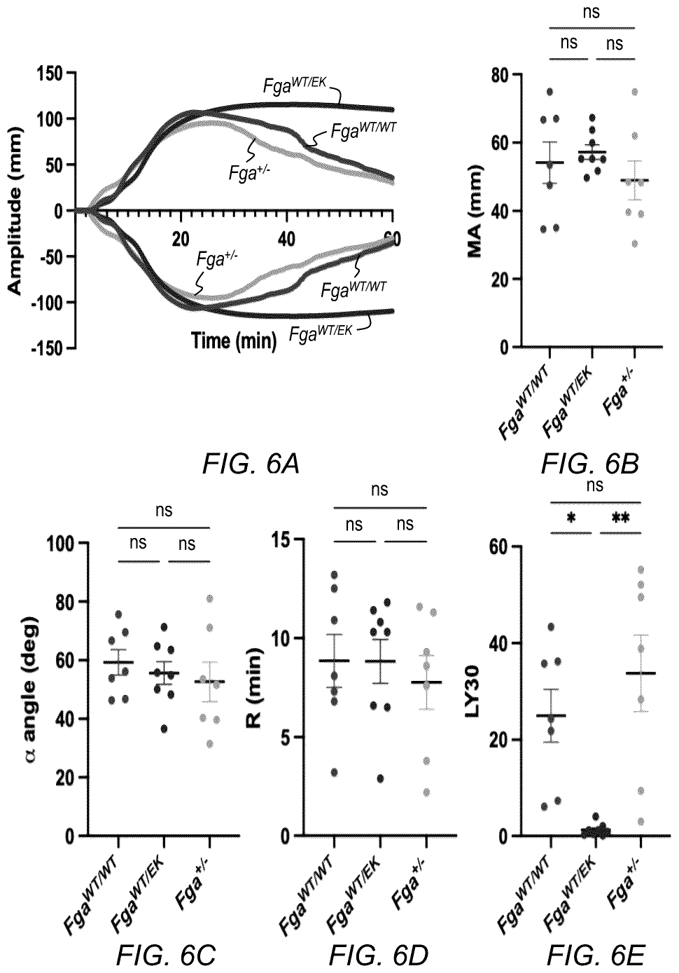

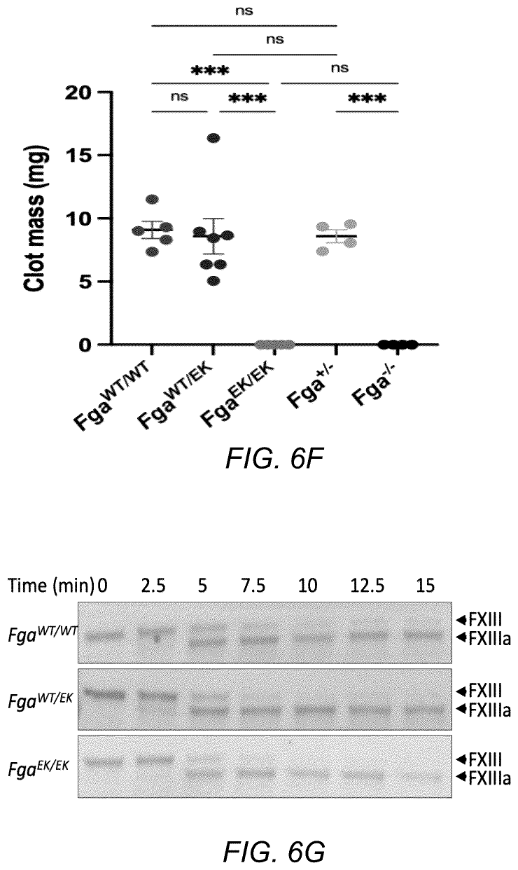

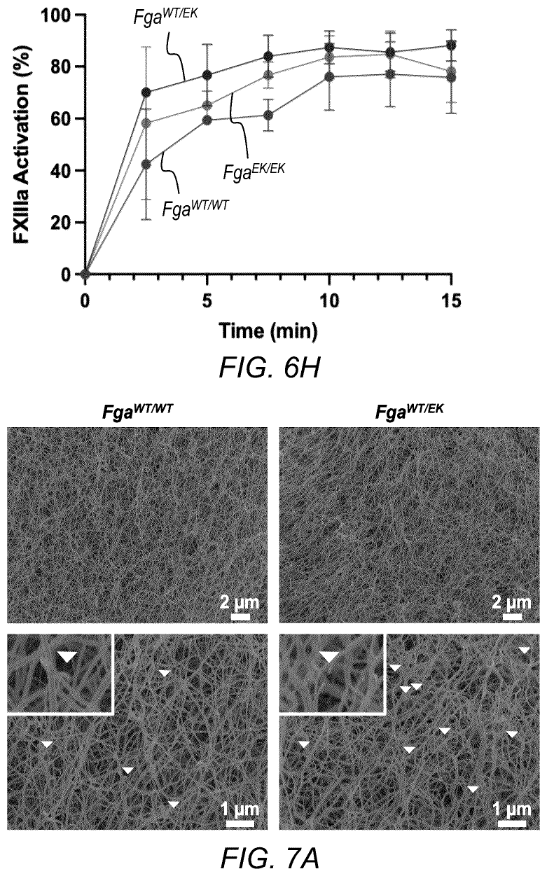

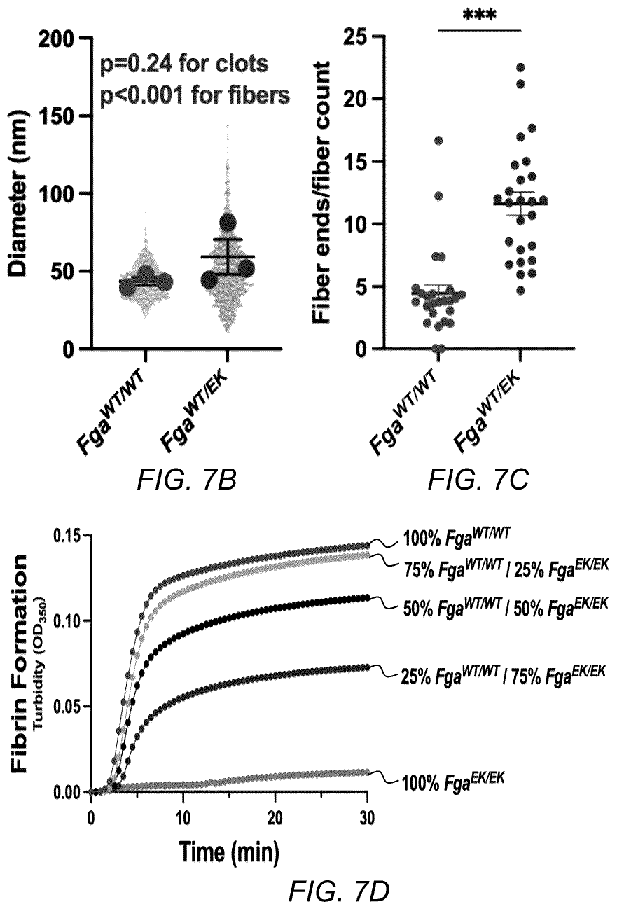

To begin to identify the mechanism by which nonpolymerizable fibrinogen suppresses venous thrombosis, we first performed standard clinical clotting tests. Fga^{WT/EK} plasma had a modest but statistically significant delay in thrombin time, compared to Fga^{WT/WT} and Fga^{+/-} plasma. In contrast, Fga^{WT/EK} plasma performed similarly to Fga^{+/-} plasma in prothrombin time (PT) and activated partial thromboplastin time (aPTT) assays with a slight delay in PT compared to Fga^{WT/WT} plasma. Fga^{EK/EK} and Fga^{-/-} plasma did not form clots. Next, thromboelastography (TEG) analyses were performed with and without tissue plasminogen activator (tPA). In the absence of tPA, robust clots formed over similar time scales in Fga^{WT/WT}, Fga^{WT/EK} and Fga^{+/-} whole blood. Fga^{EK/EK} and Fga^{-/-} whole blood did not form detectable clots by TEG (FIG. 5A). The maximum amplitude (MA) of Fga^{WT/EK} and Fga^{+/-} clots were comparable to each other but significantly lower than that of Fga^{WT/WT} clots (FIG. 5B). Intriguingly, the α angle, indicative of fibrin accumulation, was lower in Fga^{+/-} clots compared to Fga^{WT/EK} clots (FIG. 5C). No differences were observed in the clot initiation time between Fga^{WT/WT}, Fga^{WT/EK} and Fga^{+/-} clots (FIG. 5D). In the presence of tPA, no significant differences in the MA, α angle or rapid thrombelastography (R) were observed among Fga^{WT/WT}, Fga^{WT/EK} and Fga^{+/-} clots (FIGS. 6A-6D). However, the LY30 (i.e., the percent reduction 30 minutes after MA) was significantly higher in Fga^{WT/WT} and Fga^{+/-} clots compared to Fga^{WT/EK} clots (FIG. 6E), consistent with a previous report indicating reduced plasmin generation in Fga^{WT/EK} plasma (Miszta et al. (2020) Blood 135(19):1704-1717). The impact of nonpolymerizable fibrin on whole blood clot retraction was evaluated in vitro by measuring the clot mass remaining 2 hours after coagulation initiation in whole blood. While no clots were formed in Fga^{EK/EK} and Fga^{-/-} whole blood, the final masses following retraction among Fga^{WT/WT}, Fga^{WT/EK} and Fga^{+/-} clots were virtually identical (FIG. 6F). The activation rates of FXIII, which catalyzes fibrin fiber crosslinking, were similar in Fga^{WT/WT}, Fga^{WT/EK} and Fga^{EK/EK} plasma (FIG. 6G and FIG. 6H), indicating that the nonpolymerizable fibrinogen variant supported normal FXIIIa activation. To elucidate the ramifications of a nonpolymerizable fibrin on structure, clots made from Fga^{WT/WT} and Fga^{WT/EK} platelet-poor plasma were analyzed by scanning electron microscopy (SEM) (FIG. 7A). The fiber diameters of Fga^{WT/EK} clots were increased compared to those of Fga^{WT/WT} clots (FIG. 7B). Interestingly, the fibers from Fga^{WT/EK} clots appeared to have more branch points with a significant increase in the number of fiber terminal ends (FIG. 7C).

To demonstrate the effect of termination of fibrin fibril elongation in vivo, Fibrinogen^{LOCK} protein is purified from Fga^{EK/EK} and Fga^{WT/EK} mice by glycine precipitation and dialysis, as described (Hur et al. (2022) Blood 139(9):1374-88). Wild-type or Fga^{+/-} mice are administered a dose of purified fibrinogen^{LOCK} that achieves 50% and 25% of the total circulating fibrinogen pool (i.e., between ˜0.5 and 1.5 mg/mL fibrinogen^{LOCK} per mouse). Notably, in vitro turbidity assays mixing platelet-poor plasma from Fga^{EK/EK} and Fga^{WT/EK} mice indicate that introduction of fibrinogen^{LOCK} significantly reduces fibrin formation in a dose-dependent manner (FIG. 7D).

Mice undergo venous and arterial thrombotic challenges as described in Example 1 at 4-24 hours following fibrinogen^{LOCK} induction or infusion. Levels of fibrinogen^{LOCK} are also measured to determine the efficiency of LNP-Fga^{EK} mRNA delivery and expression. It is expected that reduction of fibrinogen concentration by inhibition of fibrin formation by fibrinogen^{LOCK} will result in reduced incidence and size of venous thrombi (stenosis and stasis models), and preservation of arterial blood flow (injury of carotid and cremasteric). Further, we anticipate that the ratio of fibrin degradation products to total fibrinogen will increase with imposition of fibrinogen^{LOCK}, as we expect that the formed thrombi will have an altered clot structure that is more susceptible to fibrinolysis.

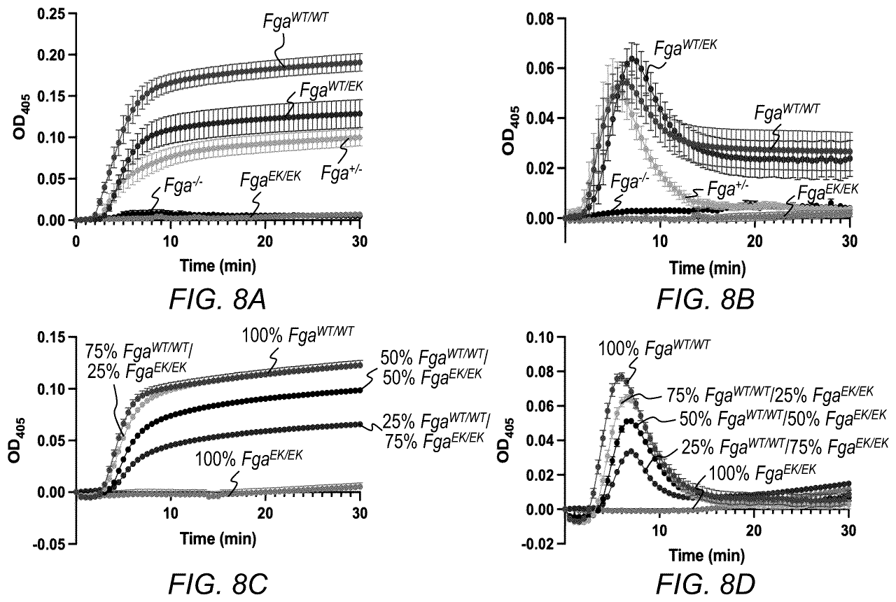

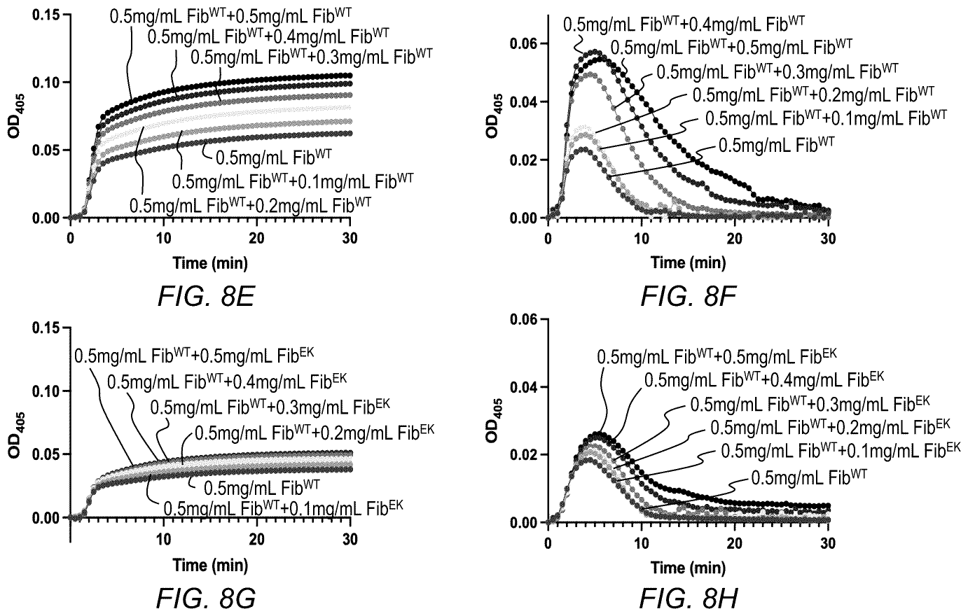

To further examine the genotype-dependent differences in fibrin formation, turbidity assays were performed. The turbidity profile of Fga^{WT/EK} plasma was lower than Fga^{WT/WT} plasma, but modestly higher than that observed for Fga^{+/-} plasma (FIG. 8A). Fga^{EK/EK} and Fga^{-/-} plasma did not display any changes in turbidity (FIG. 8A), consistent with a complete absence of fibrin formation. In the presence of tPA, initial clot formation was similar among Fga^{WT/WT}, Fga^{WT/EK} and Fga^{+/-} plasma, but Fga^{+/-} clots displayed significantly faster lysis than both Fga^{WT/WT} and Fga^{WT/EK} clots (FIG. 8B), indicating that the presence of nonpolymerizable fibrinogen stabilized the fibrin matrix that did form. When Fga^{WT/WT} and Fga^{EK/EK} plasma were mixed in different ratios, there was a dose-dependent decrease in the maximum amplitude of the turbidity profile with increasing proportions of Fga^{EK/EK} plasma (FIG. 8C). This reduction in turbidity was observed although total fibrinogen concentrations were equivalent across each condition. In the presence of tPA, increasing the ratio of Fga^{EK/EK} plasma produced clots with reduced maximum turbidity. Notably, the times at which the clots were completely degraded were similar regardless of the ratio of Fga^{EK/EK} plasma (FIG. 8D). Complementary studies were performed using a constant concentration of fibrinogen from Fga^{WT/WT} mice (Fib^{WT}) to which increasing amounts of Fib^{WT} or fibrinogen from Fga^{EK/EK} mice (Fib^{EK}) were added. Increasing Fib^{WT} resulted in a dose-dependent increase in turbidity (FIG. 8E) and a delay in fibrinolysis (FIG. 8F). In sharp contrast, supplementing increasing Fib^{EK} to a constant level of Fib^{WT} resulted in little to no change in turbidity over the same concentration range evaluated for Fib^{WT} (FIG. 8G). Of note, there was a modest delay and reduction in fibrinolysis with increasing amounts of Fib^{EK} in the reaction mixtures (FIG. 5H), indicating the presence of nonpolymerizable fibrinogen was suppressing fibrinolysis.

### Example 4: Fga^{EK} Mice Display Preserved Hemostatic Potential

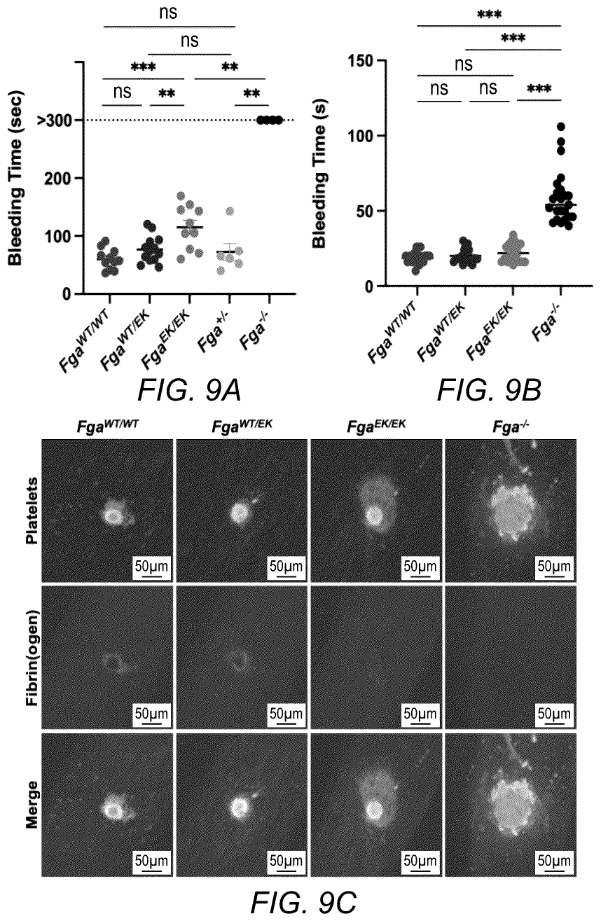

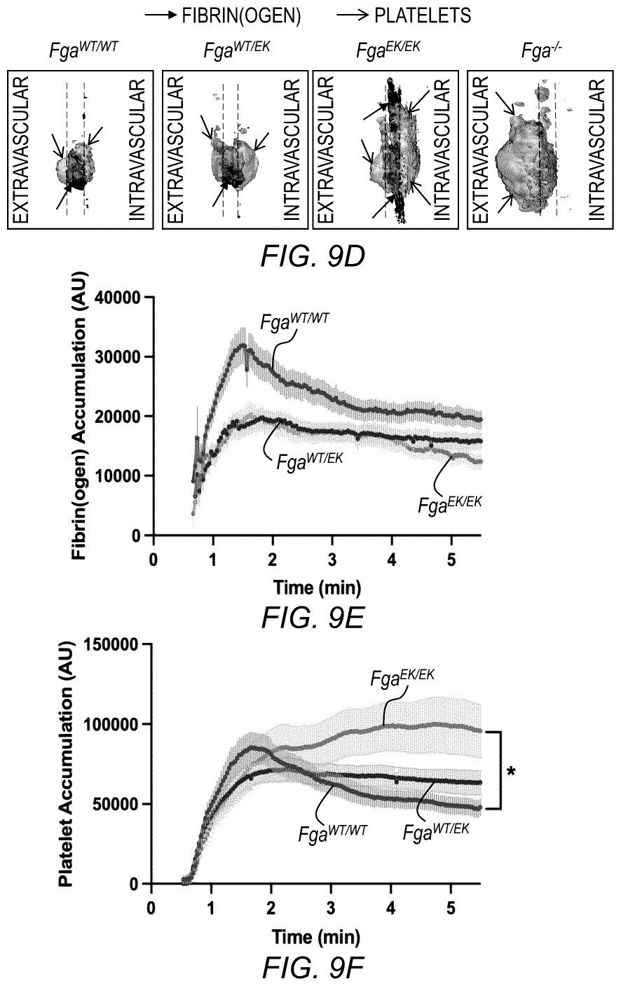

To first examine whether expression of nonpolymerizable fibrinogen alters hemostasis, tail bleeding analyses were performed. While Fga¹ mice did not stop bleeding within a 5 min observation window, Fga^{EK/EK} mice uniformly achieved hemostasis, although the bleeding times were modestly increased compared to Fga^{WT/WT} and Fga^{WT/EK} mice (FIG. 9A). Bleeding times of Fga^{WT/EK} mice were similar to that of Fga^{WT/WT} mice, indicating that nonpolymerizable fibrinogen, either alone or in combination with WT fibrinogen, does not prohibit hemostasis under these conditions.

Hemostasis was next evaluated using a laser-induced saphenous vein injury model (Paul et al. (2020) Arterioscler. Thromb. Vasc. Biol. 40(8):1891-1904; Getz et al. (2015) J. Thromb. Haemost. 13(3):417-425). No differences were observed in the bleeding times between Fga^{WT/WT}, Fga^{WT/EK} and Fga^{EK/EK} mice, while there was a significant delay in Fga-mice (FIG. 9B). In Fga^{WT/WT} and Fga^{WT/EK} mice, a defined ring of fibrin(ogen) deposition around a platelet core was observed at the injury site. In contrast, fibrinogen accumulation was diffuse and dispersed throughout the injury site in Fga^{EK/EK} mice and non-existent in Fga^{-/-} mice (FIG. 9C). Three-dimensional reconstruction of the hemostatic plug 5 minutes after laser-induced injury further indicated that clots in Fga^{WT/WT} and Fga^{WT/EK} mice were composed of tightly packed platelet plugs with equivalent platelet densities in the intravascular and extravascular spaces and a ring of fibrin at the intersection with the vessel wall. In Fga^{EK/EK} mice, both platelets and fibrinogen covered a larger area around the injury site and the fibrinogen signal was diffuse (FIG. 9D). Fga^{-/-} mice displayed a widespread platelet signal at the injury site with a substantial portion of the plug in the extravascular space. Quantification of platelets and fibrin(ogen) demonstrated an accumulation of both fibrin(ogen) and platelets at the injury site of Fga^{WT/WT} mice (FIGS. 9E-9F) that peaked and then gradually reduced over time. In Fga^{WT/EK} and Fga^{EK/EK} mice, fibrin(ogen) accumulation was reduced compared to Fga^{WT/WT} mice (FIG. 9E). Interestingly, initial platelet accumulation in Fga^{EK/EK} mice peaked at the same level and time as Fga^{WT}r and Fga^{WT/EK} mice, but remained elevated throughout the observation period (FIG. 9F).

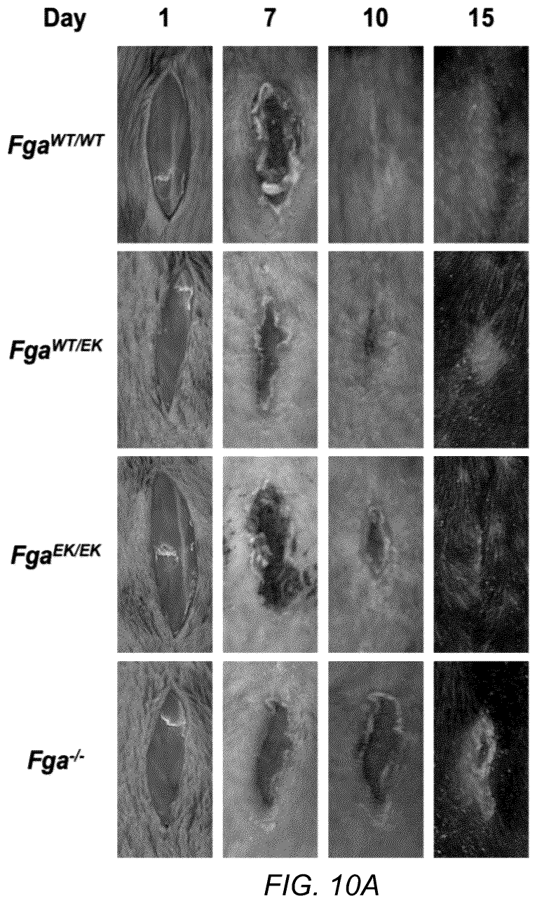

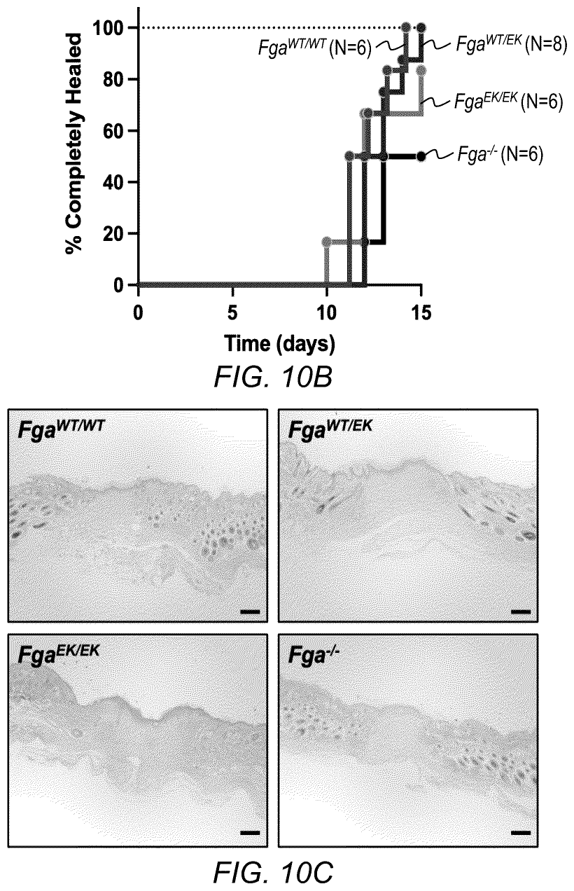

To determine the potential impact of nonpolymerizable fibrinogen on wound healing, a skin incision model was performed. No significant differences were observed among Fga^{WT/WT}, Fga^{WT/EK} and Fga^{EK/EK} mice in time to complete wound closure (FIGS. 10A-10B). Although Fga^{-/-} mice uniformly displayed a progressive reduction in the size of wound field over time, there was a significant delay in time to complete wound healing compared to Fga^{WT/WT} and Fga^{WT/EK} mice (FIG. 10B). Histological evaluation of the wound fields at the end of the 15-day observation period revealed a similar appearance across all genotypes as characterized by keratinocyte outgrowths that migrated into the wound fields and fusion of the outgrowths in mice that had completely healed (FIG. 10C).

### Example 5: Fibrinogen^{LOCK} for Mitigating Thrombosis During Endotoxemia (Acute Inflammation) and Sickle Cell Disease (Chronic Inflammatory Disease)

Suppression of fibrin of formation with fibrinogen^{LOCK} is assessed for conferring protection from thrombosis in acute LPS-induced endotoxemia. Lipopolysaccharide (LPS) injection in mice results in quantitative changes in the levels and activities of procoagulant and fibrinolytic factors that favor thrombosis. Specifically, LPS exacerbates thrombotic outcomes in both IVC and carotid artery thromboses in mouse models (Wang (2008) Thromb. Res. 123(2):355-60; Obi et al. (2017) Thromb. Haemost. 117(2):339-48; Vital et al. (2020) Cells 9(11):2473).

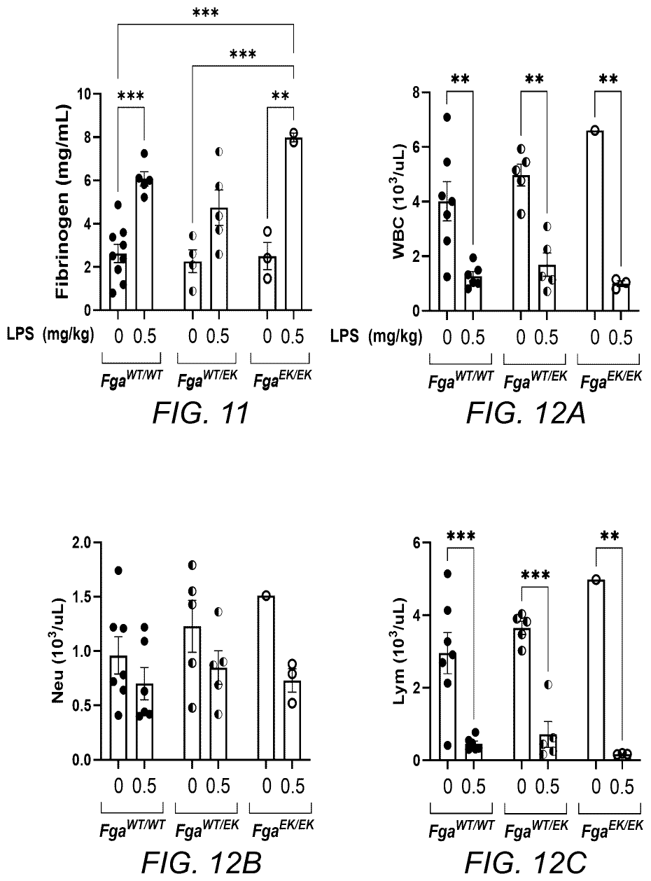

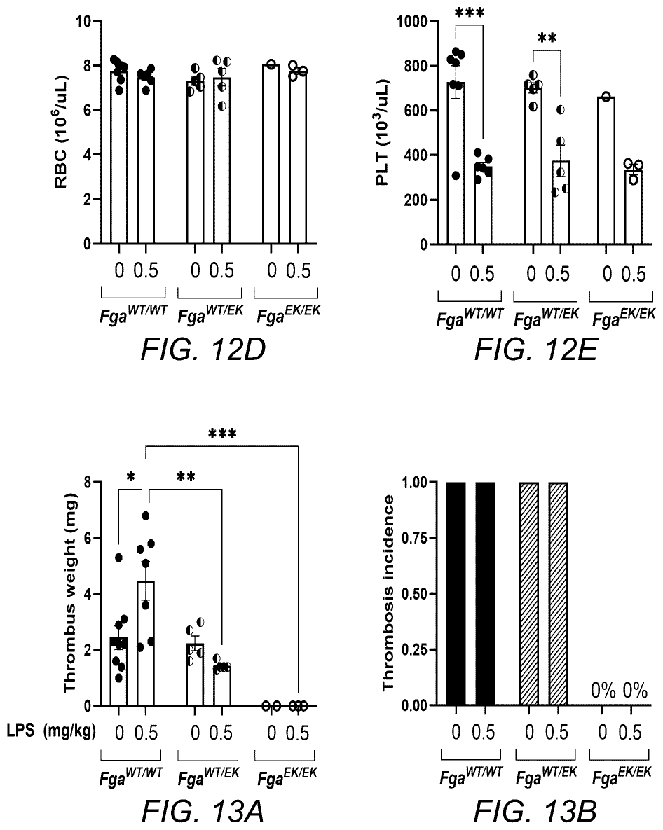

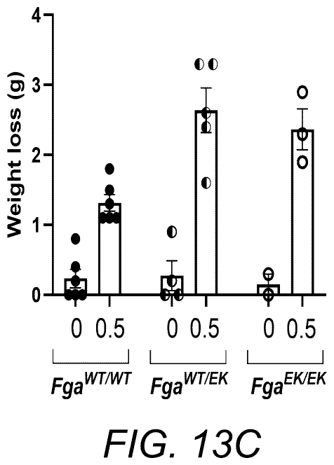

Cohorts of mice (Fga^{WT/WT}, Fga^{WT/EK}, and Fga^{EK/EK}, n=10 mice/group) were challenged with LPS (0.5 mg/kg). IVC stasis/stenosis thrombosis and FeCl₃ carotid artery thrombosis were analyzed with identical readouts (e.g., thrombus mass, thrombosis incidence) as outlined in Example 1. Further, blood draws pre- and post-thrombotic challenge were performed to assess fibrinogen levels and CBCs. This analysis indicated that LPS-induced inflammation provokes a significant increase in circulating fibrinogen in Fga^{WT/WT}, Fga^{WT/EK} and Fga^{EK/EK} mice (FIG. 11). In addition, Fga^{WT/WT}, Fga^{WT/EK}, and Fga^{EK/EK} mice display the same reactive changes in blood cells after LPS-induced inflammation (FIG. 12A-12E). Notably, Fga^{WT/EK} and Fga^{EK/EK} mice are protected from LPS-driven thrombosis (FIG. 13A-13C).

Studies have demonstrated that RBCs are not just trapped in venous clots, but instead play an active role in the pathogenesis of VTE (Wu et al. (2006) Blood 108(4)1183-8). RBCs can regulate blood flow, promote thrombin generation, and modulate thrombus size and stability (Weisel & Litvinov (2019) J. Thromb. Haemost. 17(2):271-82; Cines et al. (2014) Blood. 123(10):1596-603). In SCD, the abnormal structure, function, and quantity of RBCs are believed to enhance clot formation and contribute to VTE. Townes sickle (SS) mice are knock-in homozygous for both human α-globin and sickle β-globin (β^{S}) genes, while control Townes (AA) mice express human α-globin and normal human β-globin (β^{A}). SS mice have severe anemia, leukocytosis, enhanced thrombin generation and inflammation, and multi-organ damage consistent with the human condition (Wu et al. (2006) Blood 108(4)1183-8). It has been demonstrated that subjecting SS mice to IVC ligation or stenosis models of venous thrombosis results in rapid death within 2 hours. Thus, we developed a new model of thrombosis that allows for survival of SCD mice over the entire experimental observation period. Here, the femoral vein of mice is challenged with an electrolytic injury that drives rapid thrombus formation. Notably, these thrombi are characterized by a statistically significant increase in fibrin but not platelet content, compared to AA controls, indicating that selectively targeting fibrin(ogen) may be particularly efficacious in SCD to reduce thrombosis.

We will use the electrolytic model to determine whether targeting fibrin(ogen) is effective in reducing thrombosis in SCD. To evaluate suppression of fibrin formation in SCD, we will perform bone marrow transplants (BMT) using marrow harvested from Townes AA and SS mice into 10 weeks-old recipients (n=15 mice/group) with 10 weeks of recovery. The electrolytic injury is performed by applying a positive current (3 volts, 90 sec) to the femoral vein and analyzing thrombus formation every 10 seconds for 60 min using 100× intravital fluorescence microscopy. The accumulation of platelets (rhodamine 6G-conjugated anti-CD41) and fibrin (ALEXA FLUOR®647-conjugated anti-fibrinogen) will be quantified via relative intensity of each fluorophore over the region of the observed thrombus. As clots formed in the femoral vein are too small to weigh or perform biochemical analysis for cellular composition, we will use histomorphometry. At 1 and 24 hours after injury, the entire femoral vein will be harvested, fixed (4% paraformaldehyde in PBS), processed, and embedded in paraffin. Serial sections (5 μm) will be cut and stained with hematoxylin and eosin. The thrombus area will be measured histomorphometrically (NIH ImageTool Software) once every 10 sections (every 50 μm) over the extent of the sectioned vein. The thrombus volume will be reconstructed from these digital measurements. At 24 hours after the electrolytic injury, CBCs, fibrinogen, TAT, PAP, and FDP will be measured by ELISA. SCD-mediated changes in anticoagulant proteins, including antithrombin, protein C, and protein S will be measured by ELISA. Circulating inflammatory mediators will also be measured. We will also determine if the procoagulant changes imposed by sickle RBCs drives thrombosis by promoting the formation of pro-thrombotic fibrin clot structure. It is expected that suppression of fibrin formation with nonpolymerizable fibrinogen will reduce thrombosis even in the context of an acute inflammatory challenge (i.e., endotoxemia) or a chronic inflammatory state (i.e., SCD).

The foregoing is illustrative of the present invention, and is not to be construed as limiting thereof. The invention is defined by the following claims, with equivalents of the claims to be included therein.

1. A method for inhibiting or reducing the risk of venous or arterial thrombosis without compromising hemostasis or reducing the number and/or size of thrombi formed in a subject without compromising hemostasis, comprising administering to a subject in need thereof an effective amount of nonpolymerizable fibrinogen thereby inhibiting or reducing the risk of venous or arterial thrombosis without compromising hemostasis or reducing the number and/or size of thrombi formed in a subject without compromising hemostasis.

2. The method of claim 1, wherein the venous or arterial thrombosis or thrombi is associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts, or prostheses.

3. The method of claim 1, wherein the subject is receiving extracorporeal membrane oxygenation (ECMO) treatment.

4. The method of claim 1, wherein the subject is receiving surgery.

5. The method of claim 1, wherein the subject has a condition associated with increased risk of thrombosis or thrombi.

6. The method of claim 1, wherein the subject is human.

7. The method of claim 1, wherein the nonpolymerizable fibrinogen comprises a mutation in the thrombin cleavage site.

8. The method of claim 7, wherein the mutation comprises a E^{P6}GGGVR^{P1} to A^{P6}DDDDK^{P1} mutation.

9. The method of claim 1, further comprising administering the nonpolymerizable fibrinogen in combination with an antithrombotic agent and/or anticoagulant agent.

10. The method of claim 9, wherein the antithrombotic agent is a direct or indirect thrombin inhibitor, a Factor X inhibitor, a Factor IX inhibitor, a Factor XII inhibitor, a Factor V inhibitor, a Factor VIII inhibitor, a Factor XIII inhibitor, a Factor VII inhibitor, a tissue factor inhibitor, a profibrinolytic agent, a fibrinolytic or fibrinogenolytic agent, a carboxypeptidase B inhibitor, a platelet inhibitor, a selective platelet count reducing agent, or a Factor XI inhibitor.

11. The method of claim 1, wherein the nonpolymerizable fibrinogen is administered to the subject via systemic or parenteral administration.

12. (canceled)

13. The method of claim 1, wherein thrombi formed exhibit a heterogenous distribution of platelets and neutrophils.

14-23. (canceled)

24. A method of reducing a required dose or complementing the effect of an antithrombotic agent in the treatment of thrombosis in a subject in need thereof, comprising administering to the subject an effective amount of a nonpolymerizable fibrinogen in combination with the antithrombotic agent.

25. The method of claim 24, wherein the antithrombotic agent is a direct or indirect thrombin inhibitor, a Factor X inhibitor, a Factor IX inhibitor, a Factor XII inhibitor, a Factor V inhibitor, a Factor VIII inhibitor, a Factor XIII inhibitor, a Factor VII inhibitor, a tissue factor inhibitor, a profibrinolytic agent, a fibrinolytic or fibrinogenolytic agent, a carboxypeptidase B inhibitor, a platelet inhibitor, a selective platelet count reducing agent, or a Factor XI inhibitor.

26. The method of claim 24, wherein the nonpolymerizable fibrinogen comprises a mutation in the thrombin cleavage site.

27. The method of claim 26, wherein the mutation comprises a E^{P6}GGGVR^{P1} to A^{P6}DDDDK^{P1} mutation.

28. (canceled)

29. A pharmaceutical composition comprising an effective amount of a nonpolymerizable fibrinogen and an antithrombotic agent in admixture with a pharmaceutically acceptable carrier.

30. The pharmaceutical composition of claim 29, wherein the nonpolymerizable fibrinogen comprises a mutation in the thrombin cleavage site.

31. The pharmaceutical composition of claim 30, wherein the mutation comprises a E^{P6}GGGVR^{P1} to A^{P6}DDDDK^{P1} mutation.

32. The pharmaceutical composition of claim 29, wherein the antithrombotic agent is a direct or indirect thrombin inhibitor, a Factor X inhibitor, a Factor IX inhibitor, a Factor XII inhibitor, a Factor V inhibitor, a Factor VIII inhibitor, a Factor XIII inhibitor, a Factor VII inhibitor, a tissue factor inhibitor, a profibrinolytic agent, a fibrinolytic or fibrinogenolytic agent, a carboxypeptidase B inhibitor, a platelet inhibitor, a selective platelet count reducing agent, or a Factor XI inhibitor.
